SEQUENCE LISTING (38 sequences)

SEQ ID NO: 1 - DNA, 3174 bp, Arabidopsis thaliana
  CDS (1)..(3171)
atgaactgggcactcaacaatcatcaagaagaagaagaagagccacgaagaattgaaatttctgattccgagtcactagaaaacttgaaaagcagcgatttttatcaactgggtggtggtggtgctctgaattcgtcagaaaagccgagaaagatcgatttttggcgttcggggttgatgggttttgcgaagatgcagcagcagcaacagcttcagcattcagtggcggtgaagatgaacaataataataataacgatctaatgggtaataaaaaagggtcaactttcatacaagaacatcgagcattgttaccaaaagctttgattctgtggatcatcattgttgggtttataagcagtgggatttatcagtggatggatgatgctaataagattagaagggaagaggttttggtcagcatgtgtgatcaaagagctagaatgttgcaggatcaatttagtgttagtgttaatcatgttcatgctttggctattctcgtctccacttttcattaccacaagaacccttctgcaattgatcaggagacatttgcggagtacacggcaagaacagcatttgagagaccgttgctaagtggagtggcttatgctgaaaaagttgtgaattttgagagggagatgtttgagcggcagcacaattgggttataaagacaatggatagaggagagccttcaccggttagggatgagtatgctcctgttatattctctcaagatagtgtctcttaccttgagtcactcgatatgatgtcaggcgaggaggatcgtgagaatattttgcgagctagagaaaccggaaaagctgtcttgactagcccttttaggttgttggaaactcaccatctcggagttgtgttgacattccctgtctacaagtcttctcttcctgaaaatccgactgtcgaagagcgtattgcagccactgcagggtaccttggtggtgcgtttgatgtggagtctctagtcgagaatttacttggtcagcttgctggtaaccaagcaatagttgtgcatgtgtatgatatcaccaatgcatcagatccacttgtcatgtatggtaatcaagatgaagaagccgacagatctctctctcatgagagcaagctcgattttggagaccccttcaggaaacataagatgatatgcaggtaccaccaaaaggcaccaataccattgaatgtgctcacaactgtgccattgttctttgcgattggtttcttggtgggttatatactgtatggtgcagctatgcacatagtaaaagtcgaagatgatttccatgaaatgcaagagcttaaagtgcgagcagaagctgctgatgtcgctaaatcgcagtttcttgctaccgtgtctcacgagatcaggacaccaatgaatggcattctcggaatgcttgctatgctcctagatacagaactaagctcgacacagagagattacgctcaaaccgctcaagtatgtggtaaagctttgattgcattgataaatgaggttcttgatcgcgccaagattgaagctggaaagctggagttggaatcagtaccatttgatatccgttcaatattggatgatgtcctttctctattctctgaggagtcaaggaacaaaggcattgagctcgcggttttcgtttcagacaaagtaccagagatagtcaaaggagattcagggagatttagacagataatcataaaccttgttggaaattcggttaaattcacagagaaaggacatatctttgttaaagtccatcttgcggaacaatcaaaagatgaatctgaaccgaaaaatgcattgaatggtggagtgtctgaagaaatgatcgttgtttccaaacagtcaagttacaacacattgagcggttacgaagctgctgatggtcggaatagctgggattcattcaagcatttggtctctgaggagcagtcattatcggagtttgatatttctagcaatgttaggcttatggtttcaatcgaagacacgggtattggaatccctttagttgcacaaggccgtgtgtttatgccgtttatgcaagcagatagctcgacttcaagaaactatggaggtactggtattggtttgagtataagcaagtgtcttgttgaacttatgcgtggtcagataaatttcataagccggcctcatattggaagcacgttctggttcacggctgttttagagaaatgcgataaatgcagtgcgattaaccatatgaagaaacctaatgtggaacacttgccttctacttttaaaggaatgaaagctatagttgttgatgctaagcctgttagagctgctgtgactagataccatatgaaaagactcggaatcaatgttgatgtcgtgacaagtctcaaaaccgctgttgttgcagctgctgcgtttgaaagaaacggttctcctctcccaacaaaaccgcaacttgatatgatcttagtagagaaagattcatggatttcaactgaagataatgactcagagattcgtttattgaattcaagaaccaacggaaacgttcatcacaagtctccgaaactagctctattcgcaacaaacatcacaaattcggagttcgacagagctaaatccgcaggatttgcagatacggtaataatgaaaccgttaagagcaagcatgattggggcgtgtctgcaacaagttctcgagctgagaaaaacaagacaacaacatccagaaggatcatcacccgcaactctcaagagcttgcttacagggaagaagattcttgtggttgatgataatatagttaacaggagagtagctgcaggagctctcaagaaatttggagcagaagtggtttgtgcagagagtggtcaagttgctttgggtttgcttcagattccacacactttcgatgcttgcttcatggatattcaaatgccacagatggacggatttgaagcaactcgtcagataagaatgatggagaaggaagctaaagagaagacgaatctcgaatggcatttaccgattctagcgatgactgcggatgtgatacacgcgacctacgaggaatgtctgaaaagtgggatggatggttacgtctccaaaccttttgaagaagagaatctctataaatccgttgccaaatcattcaaacctaatcctatctcaccttcgtcgtaa

SEQ ID NO: 2 - protein, 1057 aa, Arabidopsis thaliana
MNWALNNHQEEEEEPRRIEISDSESLENLKSSDFYQLGGGGALNSSEKPRKIDFWRSGLMGFAKMQQQQQLQHSVAVKMNNNNNNDLMGNKKGSTFIQEHRALLPKALILWIIIVGFISSGIYQWMDDANKIRREEVLVSMCDQRARMLQDQFSVSVNHVHALAILVSTFHYHKNPSAIDQETFAEYTARTAFERPLLSGVAYAEKVVNFEREMFERQHNWVIKTMDRGEPSPVRDEYAPVIFSQDSVSYLESLDMMSGEEDRENILRARETGKAVLTSPFRLLETHHLGVVLTFPVYKSSLPENPTVEERIAATAGYLGGAFDVESLVENLLGQLAGNQAIVVHVYDITNASDPLVMYGNQDEEADRSLSHESKLDFGDPFRKHKMICRYHQKAPIPLNVLTTVPLFFAIGFLVGYILYGAAMHIVKVEDDFHEMQELKVRAEAADVAKSQFLATVSHEIRTPMNGILGMLAMLLDTELSSTQRDYAQTAQVCGKALIALINEVLDRAKIEAGKLELESVPFDIRSILDDVLSLFSEESRNKGIELAVFVSDKVPEIVKGDSGRFRQIIINLVGNSVKFTEKGHIFVKVHLAEQSKDESEPKNALNGGVSEEMIVVSKQSSYNTLSGYEAADGRNSWDSFKHLVSEEQSLSEFDISSNVRLMVSIEDTGIGIPLVAQGRVFMPFMQADSSTSRNYGGTGIGLSISKCLVELMRGQINFISRPHIGSTFWFTAVLEKCDKCSAINHMKKPNVEHLPSTFKGMKAIVVDAKPVRAAVTRYHMKRLGINVDVVTSLKTAVVAAAAFERNGSPLPTKPQLDMILVEKDSWISTEDNDSEIRLLNSRTNGNVHHKSPKLALFATNITNSEFDRAKSAGFADTVIMKPLRASMIGACLQQVLELRKTRQQHPEGSSPATLKSLLTGKKILVVDDNIVNRRVAAGALKKFGAEVVCAESGQVALGLLQIPHTFDACFMDIQMPQMDGFEATRQIRMMEKEAKEKTNLEWHLPILAMTADVIHATYEECLKSGMDGYVSKPFEEENLYKSVAKSFKPNPISPSS

SEQ ID NO: 3 - DNA, 3531 bp, Arabidopsis thaliana
  CDS (1)..(3528)
atgtctataacttgtgagctcttgaatcttacttcaaagaaagctaagaagtcgtcgagcagtgacaagaaatggctaaagaagcctctcttcttcctgattttgtgtggctctttggtaattgttttggttatgttcttacggttaggtagaagtcagaaggaggagacagattcttgtaatggagaagagaaagtgttgtatagacatcaaaatgtcacaagaagtgagattcatgatttggtctctttgttctctgattcagatcaggtaacatcctttgaatgtcataaggaatcaagccctggaatgtggacaaactatggtattacatgttccctgagtgtgcgttctgataaacaagagactagagggcttccctggaatcttggcttaggacattctatctcatcaacatcttgtatgtgtggtaatcttgaaccgattttacagcaacctgaaaaccttgaggaagaaaaccatgaagaagggctggagcagggtttgtcatcgtatttaagaaatgcatggtggtgtctaatccttggtgtgttagtgtgccataagatttatgtatctcattctaaagcacgaggtgagaggaaagagaaagtacatctgcaagaggctttagctccaaagaagcagcaacaacgtgctcagacttcttctagaggggctggaagatggaggaagaatatccttctccttggtattttaggaggagtttccttctctgtttggtggttttgggacactaatgaggagatcataatgaaaaggagggagactttggcaaacatgtgtgacgaacgagcacgtgttttacaagatcagttcaatgttagcttgaaccatgttcatgccttgtctattcttgtatctacatttcatcatggtaaaatcccatctgccattgatcagagaacatttgaagaatatactgagagaacaaactttgagaggccacttactagtggtgtagcgtatgctttgaaagtcccacactcagaaagagagaaatttgaaaaggagcatggatgggcaataaagaaaatggaaactgaggaccagacagttgtacaagattgtgttcctgaaaactttgatcccgcaccgattcaagacgaatacgcgccagttatatttgctcaagaaactgtttcccatattgtatcggtcgacatgatgtctggagaagaagaccgtgaaaacatcttacgggcaagggcatcaggaaaaggagtgttaacatctccatttaagcttcttaagtcaaatcatcttggtgttgtgttgacctttgctgtctatgacacgagcctaccgcctgatgctacagaagaacagcgtgttgaagcaactattgggtaccttggtgcatcatatgatatgccatcgctggtggagaaacttcttcaccaacttgccagcaaacagacaattgctgtggatgtttacgacacaactaacacttcaggtctaataaaaatgtatggctcagaaattggggatataagtgagcagcatataagtagccttgattttggtgatccatcaaggaaccatgagatgcattgcaggtttaagcataaacttcccattccctggacagcgataacaccgtcgatcttagttctggttattacttttcttgttggttatattttatatgaagccatcaaccgaattgcgacagttgaagaggattgtcagaagatgagggaactcaaagctcgtgctgaggccgctgacattgcaaagtcacagttcctagcaactgtttctcatgagatacggactccgatgaatggagttttaggaatgctgaaaatgctgatggacaccgatcttgatgcgaagcagatggactatgcgcaaactgctcatggcagtgggaaggatcttacatcactaataaatgaggttcttgatcaggcaaagattgaatccggaaggctcgagcttgaaaatgtgccttttgatatgcgttttattcttgataatgtttcatctctcctctctggcaaggcaaatgaaaaaggaattgagttggccgtttatgtttctagtcaagttcctgatgttgtagtcggtgatccgagtcggttccggcagatcattacaaacctggttggaaactcaatcaaattcacacaggaaaggggacacatatttatctcagtgcaccttgcagatgaggtaaaggagcctcttactattgaagacgcagtgctaaaacagcgactagctttaggatgcagcgagtccggtgagacagttagcgggtttcctgcggtaaatgcatggggaagctggaagaatttcaagacatgttacagtactgagagtcagaattctgatcaaatcaaattgctagttacagtggaggacactggagttggcatacctgtggatgcacaaggccgaatcttcacaccttttatgcaagccgacagttccacatcgcggacttatggtggaactggcataggtttgagtataagcaaacgtttggttgaactcatgcaaggagagatggggtttgtgagtgagcccgggataggcagtactttttcatttactggagttttcgggaaagcagaaacaaatacgtcgattactaagctggaacgattcgatctagctattcaggagtttacaggattgagagcattagttattgataacagaaacattagagcagaggtcaccaggtacgaacttcggagactgggaatatctgcagacattgtttcaagtctgagaatggcatgcacttgttgtatcagcaaattagaaaatttggctatgattctaatagacaaagacgcctggaacaaggaagaattttcagtacttgacgagttgtttacccgaagcaaagtaacctttacaagagtcccaaagatttttcttttggcaacttctgcaactcttactgagcgcagtgagatgaagtctactggtctcatcgatgaggtggtgataaagcctcttcggatgagtgtcttaatatgttgcttgcaagaaacccttgtcaatggcaagaagaggcaaccgaacagacagcgaagaaatcttggacacttgctaagagaaaaacagattctggttgtggatgataatcttgtgaacagacgagttgcagaaggtgcacttaagaaatatggagctattgttacatgcgttgagagtggcaaagctgcattggcaatgcttaagccgcctcataacttcgatgcttgcttcatggatctccagatgcctgaaatggatggatttgaagcgacaaggagagtccgtgagctggagagggaaatcaataagaaaatagcttctggagaagtttcagctgaaatgttctgtaaatttagtagttggcacgtcccgatattagcaatgacagcagatgttattcaggctactcatgaagaatgcatgaaatgtggaatggatggttatgtatcaaaaccgtttgaagaggaagtgctctacacagcggtagcaagattctttgaaccttgttaa

SEQ ID NO: 4 - protein, 1176 aa, Arabidopsis thaliana
MSITCELLNLTSKKAKKSSSSDKKWLKKPLFFLILCGSLVIVLVMFLRLGRSQKEETDSCNGEEKVLYRHQNVTRSEIHDLVSLFSDSDQVTSFECHKESSPGMWTNYGITCSLSVRSDKQETRGLPWNLGLGHSISSTSCMCGNLEPILQQPENLEEENHEEGLEQGLSSYLRNAWWCLILGVLVCHKIYVSHSKARGERKEKVHLQEALAPKKQQQRAQTSSRGAGRWRKNILLLGILGGVSFSVWWFWDTNEEIIMKRRETLANMCDERARVLQDQFNVSLNHVHALSILVSTFHHGKIPSAIDQRTFEEYTERTNFERPLTSGVAYALKVPHSEREKFEKEHGWAIKKMETEDQTVVQDCVPENFDPAPIQDEYAPVIFAQETVSHIVSVDMMSGEEDRENILRARASGKGVLTSPFKLLKSNHLGVVLTFAVYDTSLPPDATEEQRVEATIGYLGASYDMPSLVEKLLHQLASKQTIAVDVYDTTNTSGLIKMYGSEIGDISEQHISSLDFGDPSRNHEMHCRFKHKLPIPWTAITPSILVLVITFLVGYILYEAINRIATVEEDCQKMRELKARAEAADIAKSQFLATVSHEIRTPMNGVLGMLKMLMDTDLDAKQMDYAQTAHGSGKDLTSLINEVLDQAKIESGRLELENVPFDMRFILDNVSSLLSGKANEKGIELAVYVSSQVPDVVVGDPSRFRQIITNLVGNSIKFTQERGHIFISVHLADEVKEPLTIEDAVLKQRLALGCSESGETVSGFPAVNAWGSWKNFKTCYSTESQNSDQIKLLVTVEDTGVGIPVDAQGRIFTPFMQADSSTSRTYGGTGIGLSISKRLVELMQGEMGFVSEPGIGSTFSFTGVFGKAETNTSITKLERFDLAIQEFTGLRALVIDNRNIRAEVTRYELRRLGISADIVSSLRMACTCCISKLENLAMILIDKDAWNKEEFSVLDELFTRSKVTFTRVPKIFLLATSATLTERSEMKSTGLIDEVVIKPLRMSVLICCLQETLVNGKKRQPNRQRRNLGHLLREKQILVVDDNLVNRRVAEGALKKYGAIVTCVESGKAALAMLKPPHNFDACFMDLQMPEMDGFEATRRVRELEREINKKIASGEVSAEMFCKFSSWHVPILAMTADVIQATHEECMKCGMDGYVSKPFEEEVLYTAVARFFEPC

SEQ ID NO: 5 - DNA, 3111 bp, Arabidopsis thaliana
  CDS (1)..(3108)
atgagtctgttccatgtgctagggtttggtgtcaagattgggcatctcttctggatgctatgctgctggtttgtttcttggttcgttgataatgggatcgaggacaagtctggtcttttagttggctctgtcggtgatcttgagaagactaagatgactacgttgaagaagaagaacaagatgtggttctggaataagatctctagcagcggactcaagatcccgagtttctcttatcagtttcttggctctgttaaattcaacaaggcgtggtggaggaagcttgtggtggtttgggttgtcttctgggtcttggtctctatttggacgttttggtactttagctcgcaagctatggagaagaggaaagagacgctagctagtatgtgtgatgagagagctcgtatgctgcaggatcagttcaacgttagcatgaatcatgttcaagccatgtctatcttgatctcaaccttccaccatggcaagattccttctgctatcgatcagagaacattctcagagtacactgatagaacttcctttgagaggcctcttactagcggggtagcttatgctatgagggtgctccattcagagagggaagagttcgagaggcaacaaggttggactattaggaagatgtattctcttgaacaaaacccagttcacaaggatgactatgacctggaagctttggaaccatcccctgtccaagaagagtacgctccagtcatctttgctcaggacactgtttctcacgttgtttctctcgatatgctgtctgggaaagaagatcgtgaaaacgttttgcgggccaggagttcaggtaaaggggttttgacagctcctttcccattgataaagacaaatagacttggggtgatcctgacatttgcagtgtacaagagagatctcccctccaatgcaacgccaaaagagagaattgaggctactaacgggtatctcgggggagtgtttgacattgagtccctggtagaaaacttgcttcaacagctggctagcaagcaaacgattcttgtcaatgtgtacgatatcaccaatcactctcaaccgattagcatgtatggtacaaatgtgtcggctgatgggttggaacgtgttagtccactaatctttggcgatccattgagaaagcatgagatgcgttgcagatttaagcagaaaccaccatggccagtgctatcaatggtgacatcattcggtatccttgtgattgcgttacttgttgcacatataatccacgcaaccgttagtcgaatacacaaagttgaagaagattgtgataaaatgaagcagctcaagaaaaaggctgaagcagcagatgttgcaaagtcacagttccttgccactgtttcacatgaaatcagaactccaatgaatggtgttctaggaatgttgcatatgcttatggacacagagttagatgttacgcaacaggattatgttaggaccgcacaggcaagtggaaaagctttagtctcgctaataaatgaggttttggaccaagcaaagattgaatctggaaagcttgaacttgaggaggtgcggtttgatttgagaggaatattagatgatgtcctgtcactcttctctagcaagtcccaacaaaagggggtggagttggcagtatacatatctgatcgtgttccagatatgttaattggtgatcctgggaggtttcgacaaatactcacaaatcttatgggtaattccattaagttcactgagaaaggacacatctttgtaactgttcatttggtggatgagctatttgaatctatcgatggagagacagcatcatctccggaaagtacactgagtgggcttccagttgcagaccggcagaggagctgggaaaactttaaagctttcagctccaacgggcatcggagctttgaaccatctccccctgatataaacctaatcgtctcagttgaggatactggcgtagggatccctgtagaagcgcagtcccgtatttttacgcctttcatgcaagtcggaccatccatatccaggacgcatggaggcacaggaattggacttagcataagcaaatgtctagttggactgatgaagggagaaattggattctcgagtactcccaaggttgggtccacattcacatttactgctgtattttccaatgggatgcaaccagctgaaagaaagaatgacaacaaccagcccatattctcggaattccggggcatgaaagctgtggttgtggaccataggcctgcaagggcaaaagtctcgtggtaccattttcagcgtcttggaattcgagtcgaagtagttccacgtgttgaacaggctctacattatctgaagattggtactaccactgtgaatatgatactcatagagcaagaaatatggaatagggaagcagatgatttcattaaaaagctacagaaagaccctcttttcctttctcctaagttgattttgttagcaaactcagtagaatcgtcaatatcagaggctttatgcaccggtatagatcctccaatagtgatagtgaaaccattgagggcgagtatgctagcagcaactttgcagaggggattgggtattggaatcagagaaccacctcaacacaagggacctcctgctttgattctcaggaatcttctccttggtagaaaaattttaatcgtggatgataacaacgtaaacctcagagtggcagcgggagctctgaaaaagtacggagctgatgtggtctgcgctgagagtgggataaaggcaatctcattgcttaagccacctcacgagtttgatgcttgcttcatggacattcagatgccagaaatggatggatttgaagctacaaggagaatacgagatatggaagaggagatgaacaagagaataaagaatggggaggctttgatagtagagaacggtaacaaaacaagctggcatcttccggtattagcaatgacggcagatgtgatccaagcaacgcatgaggaatgtctgaagtgtggaatggatgggtatgtatcaaaaccatttgaagcagagcagctgtacagggaagtttctcgctttttcaattcgccttcagatacagaatcataa

SEQ ID NO: 6 - protein, 1036 aa, Arabidopsis thaliana
MSLFHVLGFGVKIGHLFWMLCCWFVSWFVDNGIEDKSGLLVGSVGDLEKTKMTTLKKKNKMWFWNKISSSGLKIPSFSYQFLGSVKFNKAWWRKLVVVWVVFWVLVSIWTFWYFSSQAMEKRKETLASMCDERARMLQDQFNVSMNHVQAMSILISTFHHGKIPSAIDQRTFSEYTDRTSFERPLTSGVAYAMRVLHSEREEFERQQGWTIRKMYSLEQNPVHKDDYDLEALEPSPVQEEYAPVIFAQDTVSHVVSLDMLSGKEDRENVLRARSSGKGVLTAPFPLIKTNRLGVILTFAVYKRDLPSNATPKERIEATNGYLGGVFDIESLVENLLQQLASKQTILVNVYDITNHSQPISMYGTNVSADGLERVSPLIFGDPLRKHEMRCRFKQKPPWPVLSMVTSFGILVIALLVAHIIHATVSRIHKVEEDCDKMKQLKKKAEAADVAKSQFLATVSHEIRTPMNGVLGMLHMLMDTELDVTQQDYVRTAQASGKALVSLINEVLDQAKIESGKLELEEVRFDLRGILDDVLSLFSSKSQQKGVELAVYISDRVPDMLIGDPGRFRQILTNLMGNSIKFTEKGHIFVTVHLVDELFESIDGETASSPESTLSGLPVADRQRSWENFKAFSSNGHRSFEPSPPDINLIVSVEDTGVGIPVEAQSRIFTPFMQVGPSISRTHGGTGIGLSISKCLVGLMKGEIGFSSTPKVGSTFTFTAVFSNGMQPAERKNDNNQPIFSEFRGMKAVVVDHRPARAKVSWYHFQRLGIRVEVVPRVEQALHYLKIGTTTVNMILIEQEIWNREADDFIKKLQKDPLFLSPKLILLANSVESSISEALCTGIDPPIVIVKPLRASMLAATLQRGLGIGIREPPQHKGPPALILRNLLLGRKILIVDDNNVNLRVAAGALKKYGADVVCAESGIKAISLLKPPHEFDACFMDIQMPEMDGFEATRRIRDMEEEMNKRIKNGEALIVENGNKTSWHLPVLAMTADVIQATHEECLKCGMDGYVSKPFEAEQLYREVSRFFNSPSDTES

SEQ ID NO: 7 - DNA, 1074 bp, Arabidopsis thaliana
  CDS (1)..(1071)
atgacagaactcaacttccacctcctcccaataatctccgatcgcttcacgacgacgacgacaacatcaccgtcgttctcgtcacattcttcttcttcttcttctcttctctctttcaccaaacgaagacgaaaacaccaacctttagtatcatccatacgcatggaacagtcacggtcacggaatcggaaagacaaagtcgtcgtcattttaggagcaaccggcgccggaaaatcaagactttccgtcgatctcgctactcgtttcccttcagagatcataaactccgataaaatccaagtctacgaaggattagagatcacaacgaatcagattacgttacaagaccgtcgcggcgttcctcaccatctcctcggcgtcatcaaccccgaacacggcgaactaaccgccggagagtttcgctccgccgcttcaaacgtcgtcaaagagataacttctcgtcaaaaggttccgattatcgccggtggatctaactctttcgtccacgcactcttagctcaacgattcgacccaaagttcgatcctttttcatccgggtcgtgtttaatcagctccgatttgcgttacgagtgttgtttcatctgggtcgatgtatcggagactgttctctacgagtatcttctcagaagagtcgacgaaatgatggattcaggtatgttcgaagagctgtctagattctacgacccggttaaatccggtttagaaacccggtttgggattaggaaagctataggtgtaccggagtttgacggttacttcaaagagtatccaccggagaagaagatgataaagtgggacgctttaagaaaagcggcgtacgataaggcggttgatgatatcaaaaggaacacgtggacgttagcgaagagacaagtgaagaagattgagatgctaaaagacgctggttgggaaatagaaagagttgatgcaacggcgtcgtttaaagcagtgatgatgaagagttcgtcggagaagaagtggagagagaattgggaagagcaagtgttggagccaagcgtaaagattgtgaagcggcatttggtgcaaaattag

SEQ ID NO: 8 - protein, 357 aa, Arabidopsis thaliana
MTELNFHLLPIISDRFTTTTTTSPSFSSHSSSSSSLLSFTKRRRKHQPLVSSIRMEQSRSRNRKDKVVVILGATGAGKSRLSVDLATRFPSEIINSDKIQVYEGLEITTNQITLQDRRGVPHHLLGVINPEHGELTAGEFRSAASNVVKEITSRQKVPIIAGGSNSFVHALLAQRFDPKFDPFSSGSCLISSDLRYECCFIWVDVSETVLYEYLLRRVDEMMDSGMFEELSRFYDPVKSGLETRFGIRKAIGVPEFDGYFKEYPPEKKMIKWDALRKAAYDKAVDDIKRNTWTLAKRQVKKIEMLKDAGWEIERVDATASFKAVMMKSSSEKKWRENWEEQVLEPSVKIVKRHLVQN

SEQ ID NO: 9 - DNA, 1011 bp, Arabidopsis thaliana
  CDS (1)..(1008)
atgatcatgaagatatctatggctatgtgcaaacaaccattgcctccttcgccgactttagacttccctccagcgagatttggtcccaatatgctaactctaaacccatacggtccaaaggacaaagttgtggtcatcatgggtgctaccgggacaggcaagtcacgactctccgtggatatagccacacgttttcgggctgagatcataaactcagacaagatacaagtccaccaaggtctagacattgtaaccaacaagatcacgagcgaggagagctgcggggtaccgcaccatctcctcggcgtcttgccgcctgaagccgacttaaccgccgcgaattactgtcacatggcgaatctctccattgaatccgtcctaaaccgtggaaagcttccaatcatcgttggaggttccaactcttacgtggaggctctagtggatgacaaagaaaacaagttcaggtcgagatacgactgttgttttctatgggtggacgtggcacttcccgttttgcacgggttcgtgtctgagagagttgacaagatggtggagagtggaatggttgaggaagtcagagaatttttcgacttttcgaactctgattactcaagagggatcaagaaagcaatcggatttccggagtttgacaggtttttcaggaacgagcagttcttgaatgtggaagacagagaagaactgttaagtaaagtgttggaagaaataaagaggaatacatttgagttagcttgtaggcagagagaaaagatcgaacggttgagaaaagtgaagaagtggtctattcagagagtggatgcgactccagtctttacaaagcgaaggtccaagatggatgctaacgtggcctgggagaggctcgtggctggaccaagcacagatactgtgtcgcggtttctgctggacattgccagccgacgaccgctcgtggaagcttcaacagcggttgcggccgccatggaacgcgagttgtcgcggtgtctagtggcgtga

SEQ ID NO: 10 - protein, 336 aa, Arabidopsis thaliana
MIMKISMAMCKQPLPPSPTLDFPPARFGPNMLTLNPYGPKDKVVVIMGATGTGKSRLSVDIATRFRAEIINSDKIQVHQGLDIVTNKITSEESCGVPHHLLGVLPPEADLTAANYCHMANLSIESVLNRGKLPIIVGGSNSYVEALVDDKENKFRSRYDCCFLWVDVALPVLHGFVSERVDKMVESGMVEEVREFFDFSNSDYSRGIKKAIGFPEFDRFFRNEQFLNVEDREELLSKVLEEIKRNTFELACRQREKIERLRKVKKWSIQRVDATPVFTKRRSKMDANVAWERLVAGPSTDTVSRFLLDIASRRPLVEASTAVAAAMERELSRCLVA

SEQ ID NO: 11 - DNA, 957 bp, Arabidopsis thaliana
  CDS (1)..(954)
atgaagtgtaatgacaaaatggttgtgatcatgggtgccaccggttctggcaagtcatcactctctgttgatctcgctttacattttaaagccgagatcatcaactctgacaaaatgcagttctacgatggcttgaagatcaccacgaatcaatcgaccattgaagatcgacgtggagtgccacatcaccttctcggtgaactaaacccggaggctggagaagtcacagcggcagaatttcgcgttatggcggctgaagccatctccgagattactcaacgtaaaaagctcccaatccttgccggtggatccaactcatacattcatgctctccttgcaaaatcttatgaccctgaaaactatccgttttctgatcacaagggctcaatctgctccgagttgaaatatgattgttgtttcatttggatagatgtggatcagtctgtgttattcgagtatctttctttacgtttggatcttatgatgaagtcaggtatgttcgaggagatcgctgagttccaccgctctaagaaggccccgaaagagccattggggatatggaaggctataggagtgcaagagtttgatgactacctcaaaatgtacaagtgggacaatgacatggataaatgggaccctatgagaaaggaggcttatgagaaggcggtgagagccatcaaagaaaacacatttcagctcacaaaggatcaaatcacgaagatcaacaagctgagaaatgccgggtgggacataaagaaggtggatgctacagcatcgtttcgagaggcaattagggcagccaaggaaggcgaaggtgtagccgagatgcagagaaagatatggaacaaggaagtgttggaaccgtgtgtgaagattgtcaggagccacttggaccaaccgatcaactattattattattacttttatttactaaaaagatttttaagtcttaactag

SEQ ID NO: 12 - protein, 318 aa, Arabidopsis thaliana
MKCNDKMVVIMGATGSGKSSLSVDLALHFKAEIINSDKMQFYDGLKITTNQSTIEDRRGVPHHLLGELNPEAGEVTAAEFRVMAAEAISEITQRKKLPILAGGSNSYIHALLAKSYDPENYPFSDHKGSICSELKYDCCFIWIDVDQSVLFEYLSLRLDLMMKSGMFEEIAEFHRSKKAPKEPLGIWKAIGVQEFDDYLKMYKWDNDMDKWDPMRKEAYEKAVRAIKENTFQLTKDQITKINKLRNAGWDIKKVDATASFREAIRAAKEGEGVAEMQRKIWNKEVLEPCVKIVRSHLDQPINYYYYYFYLLKRFLSLN

SEQ ID NO: 13 - DNA, 993 bp, Arabidopsis thaliana
  CDS (1)..(990)
atgaagccatgcatgacggctctaagacaagtgattcaaccattgtcgttgaacttccaaggaaacatggtggacgttccgtttttccggcgaaaagacaaggttgttttcgtcatgggagccaccggaaccggcaaatctcgtctagccattgacctagccactcgttttccggcggagattgtaaactccgacaagatccaggtctataaaggtctagacattgtgactaacaaagtcactcctgaggaaagccttggcgttcctcaccaccttctcggcaccgtccacgacacttacgaagatttcacggcggaggattttcagcgtgaagcaatcagggccgtcgagtcaatcgtccagagagaccgtgtcccgatcatagccggtggttccaattcttacatcgaggctctggtcaacgattgcgttgacttccggttaaggtataattgttgcttcttgtgggtcgacgtctctagaccggttttacactcgtttgtctcggagcgagttgataagatggttgatatgggtctcgtcgacgaggttcgccgcatcttcgatccgtcttcgtcggattactccgctggaattcgccgagccattggagttccagagctcgacgaatttctccgttcggagatgcggaattatccggcggagacgacggagagacttcttgaaacggcgatcgagaagattaaagagaacacttgtttgcttgcgtgtagacaattgcagaagattcaaaggctttacaagcagtggaagtggaacatgcaccgtgtcgacgcgacggaggtttttctccgacgaggagaagaagctgatgaggcttgggataactcagtggctcatccgagcgcactcgccgtcgaaaagttccttagttacagcgatgaccaccatttggaaggcgccaatattctcctaccggagatctctgccgttccgcctcttccagccgccgtggcggcgatttcccggtga

SEQ ID NO: 14 - protein, 330 aa, Arabidopsis thaliana
MKPCMTALRQVIQPLSLNFQGNMVDVPFFRRKDKVVFVMGATGTGKSRLAIDLATRFPAEIVNSDKIQVYKGLDIVTNKVTPEESLGVPHHLLGTVHDTYEDFTAEDFQREAIRAVESIVQRDRVPIIAGGSNSYIEALVNDCVDFRLRYNCCFLWVDVSRPVLHSFVSERVDKMVDMGLVDEVRRIFDPSSSDYSAGIRRAIGVPELDEFLRSEMRNYPAETTERLLETAIEKIKENTCLLACRQLQKIQRLYKQWKWNMHRVDATEVFLRRGEEADEAWDNSVAHPSALAVEKFLSYSDDHHLEGANILLPEISAVPPLPAAVAAISR

SEQ ID NO: 15 - DNA, 1029 bp, Arabidopsis thaliana
  CDS (1)..(1026)
atgcaacaactcatgaccttgttatcaccaccactctctcattcttctctccttcccaccgtcactaccaaattcgggtcaccacgattagtcactacgtgcatgggccatgcagggcgtaaaaatatcaaggataaggtggttctcatcacaggtacaacaggcacaggcaagtcacgcctctcagtcgatcttgccacccgtttttttcccgccgagatcataaactcggacaaaatgcaaatctacaagggattcgagattgtcacaaatctaatcccactgcatgagcaaggaggagtcccgcaccatcttctaggtcagttccacccacaagacggtgaactcacccctgcagagttccgttctttggcgacactgtccatctctaaactaatttctagcaagaaactcccgattgtagttggtggatccaactccttcaatcacgctctactcgccgagcgttttgacccggatattgatccattctctcccggatcgagtctttcaacgatctgctctgacctaaggtacaaatgttgcatcttatgggttgatgttttagagccggttctgttccaacacttgtgcaatcgtgtcgaccaaatgatcgagtcgggattggtcgagcagcttgccgaattgtacgaccctgttgtagattcgggtcgacgactaggggttcggaagacgataggagtagaggagttcgaccgatactttagagtataccctaaggagatggacaagggaatttgggacttagcgagaaaggcggcgtacgaggagacagtgaaggggatgaaagagaggacatgtcggttggtgaagaagcagaaagagaagatcatgaagctgataagaggtggttgggagattaagaggcttgacgctacggcggcaattatggctgagctgaatcaaagtacggcaaagggagaaggaaagaatgggagagagatttgggaaaaacacattgtggatgaaagtgtcgagattgtcaagaagtttttgttggaagtttag

SEQ ID NO: 16 - protein, 342 aa, Arabidopsis thaliana
MQQLMTLLSPPLSHSSLLPTVTTKFGSPRLVTTCMGHAGRKNIKDKVVLITGTTGTGKSRLSVDLATRFFPAEIINSDKMQIYKGFEIVTNLIPLHEQGGVPHHLLGQFHPQDGELTPAEFRSLATLSISKLISSKKLPIVVGGSNSFNHALLAERFDPDIDPFSPGSSLSTICSDLRYKCCILWVDVLEPVLFQHLCNRVDQMIESGLVEQLAELYDPVVDSGRRLGVRKTIGVEEFDRYFRVYPKEMDKGIWDLARKAAYEETVKGMKERTCRLVKKQKEKIMKLIRGGWEIKRLDATAAIMAELNQSTAKGEGKNGREIWEKHIVDESVEIVKKFLLEV

SEQ ID NO: 17 - DNA, 990 bp, Arabidopsis thaliana
  CDS (1)..(989)
atgaagttctcaatctcatcactgaagcaggtacaaccaatcttgtgcttcaagaacaagctatctaaggtcaacgtcaactcttttctccatcccaaagaaaaagtcatctttgtgatgggagctaccggatcgggtaagtctcgtctcgccatcgacctagcaactcgttttcaaggagagatcataaactccgacaagattcaactttacaagggcctagacgtcctaacaaacaaagtcacccctaaagaatgccgaggcgtgcctcaccacttgcttggagtattcgactccgaagccggaaacctaacggccacccagtatagccgccttgcgtcacaagcaatctcgaaactctcagcgaacaacaagcttcccatagtagccggtggatcaaactcttacatcgaagcacttgttaatcattcctcggggtttttattaaacaactacgattgttgtttcatttgggtcgacgtttccttacccgtacttaactcctttgtctcaaaacgtgtcgaccgcatgatggaagcaggattactcgaagaagtaagagaagtgttcaatccaaaagcgaattactccgtagggatacgacgagctatcggagtccccgagctccatgaatatttacgtaacgaatctctagtggaccgtgccacaaaaagtaaaatgcttgacgtagccgttaaaaatatcaaaaagaacactgagattttagcttgtcgacagttaaaaaagattcaacggcttcacaagaagtggaagatgtctatgcatcgtgttgacgccactgaggtgttcttgaaacgcaacgtagaagaacaagacgaggcttgggagaatcttgtagcgagaccaagcgagagaatcgtcgataagttttataataataataaccaactgaaaaatgatgatgttgagcactgtttggcggcatcttacggcggaggaagtggaagtagagcccacaatatgatatga

SEQ ID NO: 18 - protein, 329 aa, Arabidopsis thaliana
MKFSISSLKQVQPILCFKNKLSKVNVNSFLHPKEKVIFVMGATGSGKSRLAIDLATRFQGEIINSDKIQLYKGLDVLTNKVTPKECRGVPHHLLGVFDSEAGNLTATQYSRLASQAISKLSANNKLPIVAGGSNSYIEALVNHSSGFLLNNYDCCFIWVDVSLPVLNSFVSKRVDRMMEAGLLEEVREVFNPKANYSVGIRRAIGVPELHEYLRNESLVDRATKSKMLDVAVKNIKKNTEILACRQLKKIQRLHKKWKMSMHRVDATEVFLKRNVEEQDEAWENLVARPSERIVDKFYNNNNQLKNDDVEHCLAASYGGGSGSRAHNMI

SEQ ID NO: 19 - DNA, 993 bp, Arabidopsis thaliana
  CDS (1)..(990)
atgcaaaatcttacgtccacattcgtctctccttccatgatcccgatcacttctccgcggctgcgactgccaccaccacgatcagtagttcccatgactaccgtttgcatggaacaatcatacaagcaaaaagtggttgtgatcatgggagccaccggatcaggcaagtcatgcctctcaatcgatctagcaactcgtttctctggcgagatcgtcaattccgacaagattcaattctacgatggattgaaggtcactacgaatcaaatgagcatccttgagagatgtggagtccctcaccatctccttggtgagctccctccggatgatagcgaactaactacctccgaattccgctctttggcgtcgcggtccatctccgagattactgctcgtggaaacctcccgattatagctggtggatcaaactccttcattcatgctctccttgtcgaccgttttgaccccaaaacctatccattctcttctgagacatccatctcttccggcttgaggtacgagtgttgcttcctttgggtggatgtctcagtgtcggtcctgttcgagtacctctcgaaacgtgtcgaccagatgatggagtcagggatgttcgaggagctagccggtttctacgacccgagatattccgggtccgcaatccgagcccacgggattcacaagaccataggaatacccgagttcgaccggtacttcagcttatacccgcctgagagaaagcagaagatgtccgaatgggaccaagcaagaaagggggcgtatgacgaagctgtccaagagatcaaagagaacacatggaggcttgcgaagaagcagattgagaggatcatgaagctgaaaagcagcggatgggacattcagaggttggacgctacgccgtcatttggaagatcgtcaagagagatttgggacaatactgttttggatgaaagcatcaaggttgtgaaacgcttcttggtgaaagacaaagtgtga

SEQ ID NO: 20 - protein, 330 aa, Arabidopsis thaliana
MQNLTSTFVSPSMIPITSPRLRLPPPRSVVPMTTVCMEQSYKQKVVVIMGATGSGKSCLSIDLATRFSGEIVNSDKIQFYDGLKVTTNQMSILERCGVPHHLLGELPPDDSELTTSEFRSLASRSISEITARGNLPIIAGGSNSFIHALLVDRFDPKTYPFSSETSISSGLRYECCFLWVDVSVSVLFEYLSKRVDQMMESGMFEELAGFYDPRYSGSAIRAHGIHKTIGIPEFDRYFSLYPPERKQKMSEWDQARKGAYDEAVQEIKENTWRLAKKQIERIMKLKSSGWDIQRLDATPSFGRSSREIWDNTVLDESIKVVKRFLVKDKV

SEQ ID NO: 21 - protein, 125 aa, Saccharomyces cerevisiae
ETSVKILVVEDNHVNQEVIKRMLNLEGIENIELACDGQEAFDKVKELTSKGENYNMIFMDVQMPKVDGLLSTKMIRRDLGYTSPIVALTAFADDSNIKECLESGMNGFLSKPIKRPKLKTILTEF

SEQ ID NO: 22 - protein, 118 aa, Escherichia coli
NDDMMILVVDDHPINRRLLADQLGSLGYQCKTANDGVDALNVLSKNHIDIVLSDVNMPNMDGYRLTQRIRQLGLTLPVIGVTANALAEEKQRCLESGMDSCLSKPVTLDVIKQSLTLY

SEQ ID NO: 23 - DNA, 31 bp, Artificial Sequence
  Description of Artificial Sequence Designed oligonucleotide primer for PCR
accatgaactgggcactcaacaatcatcaag

SEQ ID NO: 24 - DNA, 30 bp, Artificial Sequence
  Description of Artificial Sequence Designed oligonucleotide primer for PCR
ggattacgacgaaggtgagataggattagg

SEQ ID NO: 25 - DNA, 24 bp, Artificial Sequence
  Description of Artificial Sequence Designed oligonucleotide primer for PCR
tcagatatgaactgggcactcaac

SEQ ID NO: 26 - DNA, 24 bp, Artificial Sequence
  Description of Artificial Sequence Designed oligonucleotide primer for PCR
ctcaatgcttttgttccttgactc

SEQ ID NO: 27 - DNA, 32 bp, Artificial Sequence
  Description of Artificial Sequence Designed oligonucleotide primer for PCR
tccccgcggaaaatgttcttacggttaggtag

SEQ ID NO: 28 - DNA, 33 bp, Artificial Sequence
  Description of Artificial Sequence Designed oligonucleotide primer for PCR
tcggtcgacttatgattctgtatctgaaggcga

SEQ ID NO: 29 - DNA, 32 bp, Artificial Sequence
  Description of Artificial Sequence Designed oligonucleotide primer for PCR
gatcccagctagctagggccctaccgcgggga

SEQ ID NO: 30 - DNA, 32 bp, Artificial Sequence
  Description of Artificial Sequence Designed oligonucleotide primer for PCR
ctagtccccgcggtagggccctagctagctgg

SEQ ID NO: 31 - DNA, 31 bp, Artificial Sequence
  Description of Artificial Sequence Designed oligonucleotide primer for PCR
tccccgcggaaaatgtctataacttgtgagc

SEQ ID NO: 32 - DNA, 33 bp, Artificial Sequence
  Description of Artificial Sequence Designed oligonucleotide primer for PCR
ctagctagcttaacaaggttcaaagaatcttgc

SEQ ID NO: 33 - DNA, 33 bp, Artificial Sequence
  Description of Artificial Sequence Designed oligonucleotide primer for PCR
tccccgcggaaaatgaaagcacgaggtgagagg

SEQ ID NO: 34 - DNA, 32 bp, Artificial Sequence
  Description of Artificial Sequence Designed oligonucleotide primer for PCR
ctagctagcttaacaaggttcaaagaatttgc

SEQ ID NO: 35 - DNA, 30 bp, Artificial Sequence
  Description of Artificial Sequence Designed oligonucleotide primer for PCR
aaaatgaagtgtaatgacaaaatggttgtg

SEQ ID NO: 36 - DNA, 27 bp, Artificial Sequence
  Description of Artificial Sequence Designed oligonucleotide primer for PCR
gacatttgttttgcggtgatattagtc

SEQ ID NO: 37 - DNA, 34 bp, Artificial Sequence
  Description of Artificial Sequence Designed oligonucleotide primer for PCR
ttatacatatgaagccatgcatgacggctctaag

SEQ ID NO: 38 - DNA, 28 bp, Artificial Sequence
  Description of Artificial Sequence Designed oligonucleotide primer for PCR
cgggatcctcaccgggaaatcgccgcca

(19) United States
(12) Patent Application Publication
(10) Pub. No.: US 2005/0164370 A1
(43) Pub. Date: Jul. 28, 2005

(54) Transformed cell co-expressing cytokinin receptor and cytokinin biosynthesis enzyme

(75) Inventors: Tatsuo Kakimoto, Toyonaka-shi (JP); Masayuki Higuchi, Okayama-shi (JP)

Correspondence Address: SUGHRUE MION, PLLC, 2100 PENNSYLVANIA AVENUE, N.W., SUITE 800, WASHINGTON, DC, 20037, US

(73) Assignee: SUMITOMO CHEMICAL COMPANY, LIMITED

(21) Appl. No.: 10/960,929

(22) Filed: Oct. 12, 2004

(30) Foreign Application Priority Data
Oct. 10, 2003 (JP) 2003-351758

(51) Int. Cl. C12N 1/18

(52) U.S. Cl. 435/254.2

(57) ABSTRACT

The present invention provides a transformed cell co-expressing a cytokinin receptor and a cytokinin biosynthesis enzyme and a method for analyzing the activity of controlling biosynthesis of cytokinins using such a transformed cell.

### BACKGROUND OF THE INVENTION

1. Field of the Invention

The present invention relates to a method for analyzing agonist-activity and antagonist-activity of an examinee to cytokinin receptor.

2. Description of the Related Art

Cytokinins are plant hormones relevant to cell division and differentiation of higher plants and are known as important physiologically active substances having functions of inducing division of cells of higher plants, differentiating callus or pith to stems and leaves, preventing ethiolating and defoliating, preventing dropping of fruits, breaking dominance of apical buds, and the like [Cytokinins: Chemistry, Activity, and Function, CRC press (1994)]. In recent years, it has been found that the key enzyme of cytokinin biosynthesis of plants is isopentenyltransferase and that this enzyme transfers an isopentenyl group of dimethylallyl diphosphate to adenosine-5′-triphosphate and/or adenosine-5′-diphosphate [Plant & Cell Physiol. (2001) 42,677-685; J. Biol. Chem. (2001) 276, 2.6405-26410; and WO2002072818A1].

Substances having the activity of controlling biosynthesis of cytokinins can be used as plant growth regulators, for example, agents for preventing the dropping of fruits such as apples and oranges, agents for preventing plants such as rice plants and wheat from falling down by regulating the height of such plants, agents for increasing sweetness of fruits after harvest, and agents for suppressing axillary buds of tobaccoes or roses.

As a method for finding such substances having the activity of controlling biosynthesis of cytokinins, a method, in which examinee substances are directly sprayed to plants to observe and evaluate the physiological changes of the plants, can be employed.

The aforementioned method has a problem in that it is necessary to prepare the examinee substances in amounts sufficient to directly spray them to plants and it takes a great deal of time to grow the plants and to observe and evaluate the physiological changes of the plants after spraying of the examinee substances. Therefore, there has been a demand for development of various methods for quickly finding substances having the activity of controlling biosynthesis of cytokinins with small amounts of examinee substances.

### SUMMARY OF THE INVENTION

Inventors of the present invention have intensively investigated in such situations and they have found a transformed cell co-expressing a cytokinin receptor and a cytokinin biosynthesis enzyme for use in analyzing the activity of controlling biosynthesis of cytokinins of examinee substances and quickly searching substances having the activity of controlling biosynthesis of cytokinins with small amounts of samples, leading to the completion of the present invention.

The present invention provides:

1. A cell transformed with a polynucleotide comprising a nucleotide sequence encoding an amino acid sequence of a cytokinin receptor and a polynucleotide comprising a nucleotide sequence encoding an amino acid sequence of a cytokinin biosynthesis enzyme (hereinafter sometimes referred to as a transformed cell of the present invention);

2. The transformed cell according to claim 1, wherein said cytokinin receptor is selected from the group consisting of:
(a) a cytokinin receptor having the amino acid sequence represented by SEQ ID No: 2;
(b) a cytokinin receptor having the amino acid sequence represented by SEQ ID No: 4;
(c) a cytokinin receptor having the amino acid sequence represented by SEQ ID No: 6;
(d) a partially transmembrane region-deleted type cytokinin receptor;
(e) a cytokinin receptor having the amino acid sequence represented by amino acids 196 to 1176 of SEQ ID No: 4;
(f) a cytokinin receptor having the amino acid sequence represented by amino acids 50 to 1176 of SEQ ID No: 4;
(g) a cytokinin receptor having the amino acid sequence represented by amino acids 32 to 1036 of SEQ ID No: 6;
(h) a chimera-type cytokinin receptor comprising an extracellular region of the cytokinin receptor, transmembrane regions of the cytokinin receptor, a histidine kinase region of the cytokinin receptor, and a receiver region of the histidine kinase, wherein said extracellular region, said transmembrane regions and said histidine kinase region are homogeneous to one another and the receiver region is heterogeneous thereto;
(i) a cytokinin receptor having the amino acid sequence of (a), (b). (c), (e), (f), or (g) with deletion, substitution, or addition of one or a plurality of amino acids; and
(j) a cytokinin receptor having an amino acid sequence encoded by a nucleotide sequence of a polynucleotide, wherein said polynucleotide hybridizes under a stringent condition to a polynucleotide that has a nucleotide sequence complementary to a nucleotide sequence encoding the amino acid sequence represented by SEQ ID Nos: 2, 4, or 6;

3. The transformed cell according to claim 1, wherein said cytokinin biosynthesis enzyme is a isopentenyltransferase;

4. The transformed cell according to claim 1, wherein said cytokinin biosynthesis enzyme is selected from the group consisting of:
(a) a cytokinin biosynthesis enzyme having the amino acid sequence represented by SEQ ID No: 8;
(b) a cytokinin biosynthesis enzyme having the amino acid sequence represented by SEQ ID No: 10;
(c) a cytokinin biosynthesis enzyme having the amino acid sequence represented by SEQ ID No: 12;
(d) a cytokinin biosynthesis enzyme having the amino acid sequence represented by SEQ ID No: 14;
(e) a cytokinin biosynthesis enzyme having the amino acid sequence represented by SEQ ID No: 16;
(f) a cytokinin biosynthesis enzyme having the amino acid sequence represented by SEQ ID No: 18;
(g) a cytokinin biosynthesis enzyme having the amino acid sequence represented by SEQ ID No: 20;
(h) a cytokinin biosynthesis enzyme having the amino acid sequence represented by SEQ ID Nos: 8, 10, 12, 14, 16, 18, or 20 with deletion, substitution, or addition of one or a plurality of amino acids; and
(i) a cytokinin biosynthesis enzyme having an amino acid sequence encoded by a nucleotide sequence of a polynucleotide, wherein said polynucleotide hybridizes under a stringent condition to a polynucleotide that has a nucleotide sequence complementary to a nucleotide sequence encoding the amino acid sequence represented by SEQ ID Nos: 8, 10, 12, 14, 16, 18, or 20;

5. The transformed cell according to claim 1, wherein said cell is yeast; and

6. The transformed cell according to claim 1, wherein said cell is budding yeast.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

### DETAILED DESCRIPTION OF THE PREFERRED EMBODIMENTS

Hereinafter, the present invention will be described in detail.

A cytokinin biosynthetic pathway in plants has been considered to include isopentenylation of some kind of adenine structure, because cytokinins contain an isopentenyl group or a hydroxylated isopentenyl group. In recent years, it has been found that the key step of cytokinin biosynthesis in plants is isopentenylation of adenosine-540 -triphosphate and/or adenosine-540 -diphosphate with dimethylallyl diphosphate as isopentenyl donor, more concretely, transfer of an isopentenyl group from dimethylallyl diphosphate to N6 of adenosine-5′-triphosphate and/or adenosine-5′-diphosphate. An enzyme which can catalyze the isopentenylation, more concretely, an enzyme which can catalyze the transfer of the isopentenyl group, such as isopentenyltransferase, is a representative example of cytokinin biosynthesis enzyme to be used in the present invention.

Practical examples of the cytokinin biosynthesis enzymes are a cytokinin biosynthesis enzyme having the amino acid sequence represented by SEQ ID No: 8; a cytokinin biosynthesis enzyme having the amino acid sequence represented by SEQ ID No: 10; a cytokinin biosynthesis enzyme having the amino acid sequence represented by SEQ ID No: 12; a cytokinin biosynthesis enzyme having the amino acid sequence represented by SEQ ID No: 14; a cytokinin biosynthesis enzyme having the amino acid sequence represented by SEQ ID No: 16; a cytokinin biosynthesis enzyme having the amino acid sequence represented by SEQ ID No: 18; a cytokinin biosynthesis enzyme having the amino acid sequence represented by SEQ ID No: 20; a cytokinin biosynthesis enzyme having the amino acid sequence represented by SEQ ID Nos: 8, 10, 12, 14, 16, 18, or 20 with deletion, substitution, or addition of one or a plurality of amino acids; a cytokinin biosynthesis enzyme having an amino acid sequence encoded by a nucleotide sequence of a polynucleotide, wherein said polynucleotide hybridizes under a stringent condition to a polynucleotide that has a nucleotide sequence complementary to a nucleotide sequence encoding the amino acid sequence represented by SEQ ID Nos: 8,10, 12, 14, 16, 18, or 20; a cytokinin biosynthesis enzyme having an amino acid sequence encoded by a nucleotide sequence of a polynucleotide, wherein said polynucleotide hybridizes under a stringent condition to a polynucleotide that has a nucleotide sequence complementary to the nucleotide sequence represented by SEQ ID Nos: 7, 9, 11, 13, 15, 17, or 19; and the like. Incidentally, the phrase, “a plurality of amino acids”, means more particularly about 2 to 20 amino acids and for example, 2 to 10 amino acids and 2 to 5 amino acids may be exemplified. Also, the phrase, “the amino acid sequence . . . with deletion, substitution, or addition of one or a plurality of amino acids”, means as examples those having the amino acid sequences of which 80% or higher, preferably 90% or higher, and more preferably 95% or higher are identical with the sequences of the amino acids before the deletion, substitution or addition of amino acids (i.e. amino acid sequence identity of 80% or higher, particularly 90% or higher, and more preferably 95% or higher).

Those phrases, “the amino acid sequence . . . with deletion, substitution, or addition of one or a plurality of amino acids” or “the amino acid sequences of which 80% or higher . . . are identical” include amino acid sequences of the proteins provided by the intracellular processing to which proteins having the amino acid sequences represented by SEQ ID Nos: 8, 10, 12, 14, 16, 18, or 20 are subjected, and the natural variations caused by differences in type of organisms from which the proteins are derived, differences in individual bodies, differences in tissues, and the like.

A cytokinin receptor is a protein having functions of controlling the propagation and differentiation of cells of higher plants based on the intracellular signal transduction mechanism so-called Two-Component regulatory system (or Histidine to Aspartic acid phosphorelay system) while being specifically bonded with cytokinins such as purine type cytokinins, e.g. kinetine, zeatine, and the like, and urea type cytokinins, e.g. N-phenyl-N′-(4-pyridyl)urea and the like. The cytokinin receptor to be used in the present invention belongs to the histidine kinase family and is protein composed of extracellular regions, transmembrane regions, histidine kinase regions (regions having histidine kinase activity in the cell and holding Histidine residue to be an active site), and receiver regions (regions having a reception part for phosphate group transfer and holding Aspartic acid residue to be an active site).

Practical examples of the cytokinin receptor are a cytokinin receptor having the amino acid sequence represented by SEQ ID No: 2; a cytokinin receptor having the amino acid sequence represented by SEQ ID No: 4; a cytokinin receptor having the amino acid sequence represented by SEQ ID No: 6; a cytokinin receptor having the amino acid sequence represented by SEQ ID No: 2 with deletion, substitution, or addition of one or a plurality of amino acids; a cytokinin receptor having the amino acid sequence represented by SEQ ID No: 4 with deletion, substitution, or addition of one or a plurality of amino acids; a cytokinin receptor having the amino acid sequence represented by SEQ ID No: 6 with deletion, substitution, or addition of one or a plurality of amino acids; a cytokinin receptor having an amino acid sequence encoded by a nucleotide sequence of a polynucleotide, wherein said polynucleotide hybridizes under a stringent condition to a polynucleotide that has a nucleotide sequence complementary to a nucleotide sequence encoding the amino acid sequences represented by SEQ ID No: 2; a cytokinin receptor having an amino acid sequence encoded by a nucleotide sequence of a polynucleotide, wherein said polynucleotide hybridizes under a stringent condition to a polynucleotide that has a nucleotide sequence complementary to a nucleotide sequence encoding the amino acid sequences represented by SEQ ID No: 4; a cytokinin receptor having an amino acid sequence encoded by a nucleotide sequence of a polynucleotide, wherein said polynucleotide hybridizes under a stringent condition to a polynucleotide that has a nucleotide sequence complementary to a nucleotide sequence encoding the amino acid sequences represented by SEQ ID No: 6; a cytokinin receptor having an amino acid sequence encoded by a nucleotide sequence of a polynucleotide, wherein said polynucleotide hybridizes under lo a stringent condition to a polynucleotide that has a nucleotide sequence complementary to the nucleotide sequence represented by SEQ ID Nos: 1, 3, or 5; cytokinin receptors of partially transmembrane region-deleted type which will be described later; chimera-type cytokinin receptors which will be described later; and the like. Incidentally, the phrase, “a plurality of amino acids”, means more particularly about 2 to 20 amino acids and for example, 2 to 10 amino acids and 2 to 5 amino acids may be exemplified. Also, the phrase, “the amino acid sequences . . . with deletion, substitution, or addition of one or a plurality of amino acids”, means as examples those having the amino acid sequences of which 80% or higher, preferably 90% or higher, and more preferably 95% or higher are identical with the sequences of the amino acids before the deletion, substitution or addition of amino acids (i.e. amino acid sequence identity of 80% or higher, particularly 90% or higher, and more preferably 95% or higher).

Those phrases, “the amino acid sequence . . . with deletion, substitution or addition of amino acids” or “the amino acid sequences of which 80% or higher . . . are identical” include amino acid sequences of the proteins provided by the intracellular processing to which proteins having the amino acid sequences represented by SEQ ID Nos: 2, 4, and 6 are subjected, and the natural variations caused by differences in type of organisms from which the proteins are derived, differences in individual bodies, differences in tissues, and the like.

The phrase, “sequence identity”, in the present invention means the identity and homology between two DNA sequences and between two protein sequences. The sequence identity may be determined by comparing two sequences aligned in the optimum states in a region of the sequences of the comparison objects. The DNA or proteins, the comparison objects, may have addition or deletion (e.g. gap and the like) in the optimum alignment of two sequences. Regarding such sequence identity, computation may be performed using, for example, Vector NTI by producing alignment by utilizing Clustal W algorithm [Nucleic Acid Res., 22 (22): 4673-4680 (1994)]. Incidentally, the sequence identity may be measured by a sequence analysis software, practically Vector NTI, GENETYX-MAC and analysis tools provided in public databases. The aforementioned public databases may be, in general, accessible in, for example, the webpage (http://www.ddbj.nig.ac.jp) of the DNA Data Bank of Japan (the international databank operated within the Center for Information Biology and DNA Data Bank of Japan).

Regarding the phrase, “hybridizes under a stringent condition”, hybridization in this case may be performed according to a conventional method described in, for example, Molecular Cloning 2nd edition, written by Sambrook J., Frisch E. P., Maniatis T., issued by Cold Spring Harbor Laboratory press. As the “stringent condition”, there can be mentioned, for example, the conditions under which a hybrid is formed in a solution of 6×SSC (a solution containing 1.5 M NaCl and 0.15 M trisodium citrate is defined as 10×SSC) at 65° C. and then the hybrid is washed with 1×SSC at a room temperature. The salt concentration in the washing step may be selected, for example, from the condition of 1×SSC at a room temperature (a low stringent condition) to 0.1×SSC at a room temperature (a high stringent condition). The temperature in the washing step may be selected, for example, from a room temperature (a low stringent condition) to 68° C. (a high stringent condition). Further, both of the salt concentration and the temperature may be changed.

Production of a cell that is transformed with a polynucleotide comprising a nucleotide sequence encoding an amino acid sequence of a cytokinin receptor and a polynucleotide comprising a nucleotide sequence encoding an amino acid sequence of a cytokinin biosynthesis enzyme will be described below.

A cell transformed with a polynucleotide comprising a nucleotide sequence encoding an amino acid sequence of a cytokinin receptor and a polynucleotide comprising a nucleotide sequence encoding an amino acid sequence of a cytokinin biosynthesis enzyme may be obtained by introducing into a host cell, for example, in the following manner, a polynucleotide comprising a nucleotide sequence encoding an amino acid sequence of a cytokinin receptor and a polynucleotide comprising a nucleotide sequence encoding an amino acid sequence of a cytokinin biosynthesis enzyme.

(1) Preparation of cDNA

At first, the total RNA is prepared from plants such as higher plants according to the method described in, for example, Molecular Cloning 2nd edition written by J., Sambrook, E., P., Frishch, T., Maniastis.

Concretely, for example, after a part of tissues are sampled from a higher plant, the tissues are frozen in liquified nitrogen and successively physically milled using a mortar and pestle or the like. The total RNA may then be obtained by methods in which (a) the resulting milled product is mixed with a solution containing guanidine hydrochloride with phenol or a solution containing SDS with phenol to obtain the total RNA or (b) the resulting milled product is mixed with a solution containing guanidine thiocyanate and further with CsCl and then subjected to centrifugal separation to obtain the total RNA. Examples of the higher plant include a monocotyledonous plant, e.g. rice, corn, barley, wheat and the like and a dicotyledonous plant, e.g. tobacco, soybean, Arabidopsis, and the like. For the aforementioned process, commercialized kits such as ISOGEN (produced by Nippon Gene Co.), RNeasy Total RNA Purification Kit (produced by QIAGEN Co.), and the like may be employed.

Next, mRNA is prepared from the total RNA. For example, the preparation may be carried out by a method utilizing the hybridization of oligo-dT chains bonded with cellulose or latex and poly(A) chains of mRNA. For the operation, for example, commercialized kits such as mRNA Purification Kit (produced by Amersham Pharmacia Co.). OLIGOTEX™ dT30 super (Oligo(dT) latex beads) (produced by Takara Shuzo Co. Ltd.), and the like may be employed.

Further, cDNA is produced using the mRNA (mRNA having poly(A) chains) may be prepared in the following manner. For example, oligo-dT chains or random primers are annealed with mRNA and then reacted with reverse transcriptase to produce cDNA. Further, the cDNA is reacted with, for example, RNaseH, DNA polymerase I to produce double-stranded cDNA. For the operation, for example, the following commercialized kits may be employed: SMARTS PCR cDNA Synthesis Kit (produced by Clontech Co.), cDNA Synthesis Kit (produced by Takara Shuzo Co. Ltd.) cDNA Synthesis Kit (produced by Amersham Pharmacia Co.), ZAP-cDNA Synthesis Kit (produced by Stratagene Co.) and the like.

(2) Cloning

A polynucleotide comprising a nucleotide sequence encoding an amino acid sequence of a cytokinin receptor may be obtained by a polymerase chain reaction (hereinafter referred as to PCR) from the produced cDNA using, for example, a polynucleotide having partial nucleotide sequence of the nucleotide sequence of SEQ ID Nos: 1, 3 or 5 as a primer or by a hybridization method using a polynucleotide having partial nucleotide sequence of the nucleotide sequence of SEQ ID Nos: 1, 3 or 5 as a probe. In a similar manner, a polynucleotide comprising a nucleotide sequence encoding an amino acid sequence of a cytokinin biosynthesis enzyme may be obtained by a PCR using, for example, a polynucleotide having partial nucleotide sequence of the nucleotide sequence of SEQ ID Nos: 7, 9, 11, 13, 15, 17 or 19 as a primer or by a hybridization method using a polynucleotide having partial nucleotide sequence of the nucleotide sequence of SEQ ID Nos: 7, 9, 11, 13, 15, 17 or 19 as a probe.

In the case of employing PCR, polynucleotides usable as a primer set is those designed and synthesized based on the nucleotide sequences of about 20 bp to 40 bp, for example, the nucleotide sequences selected respectively from 5′-non-coding regions and 340 -non-coding regions of the nucleotide sequence represented by SEQ ID Nos: 1, 3, or 5. Examples of the primer set are sets of the polynucleotide of nucleotide sequence represented by SEQ ID No: 23 and the polynucleotide of nucleotide sequence represented by SEQ ID No: 24. The PCR solution to be used may be prepared by adding reaction solutions instructed by the kit to 250 ng of cDNA. The conditions of the PCR may properly be changed depending on the primer set to be used and, for example, concrete conditions include as follows: keeping at 94° C. for 2 minutes, and then at about 8° C. for 3 minutes, and further repeating 40 cycles each of -which comprises steps of keeping at 94° C. for 30 seconds, at 55° C. for 30 seconds, and at 72° C. for 4 minutes; and repeating 5 to 10 cycles each of which comprises steps of keeping at 94° C. for 5 seconds and at 72° C. for 4 minutes, and further repeating about 20 to 40 cycles each of which comprises steps of keeping at 94° C. for 5 seconds and at 70° C. for 4 minutes. For the operation, the following commercialized kits may be, for example, employed: HERCULASE™ (DNA polymerase)(produced by Stratagene Co., Ltd.), DNA polymerase contained in Advantage cDNA PCR Kit (Clontech Co.), TAKARA Ex Taq (Takara Shuzo Co., Ltd.), PLATINUM™ PCR SUPER (thermostable DNA polymerase in a PCR reaction mix) (Lifetech Oriental Co.), and the like.

In the case of employing hybridization, cloning may be carried out according to a method described in, for example, “Cloning and Sequence”, Experimental Manual of Plant Biotechnology (edited by Watanabe and Sugiura, Noson Bunka Publisher, 1989)

The probe to be used may be obtained by preparing polynucleotide (with the chain length of about 200 nucleotides to 500 nucleotides) having partial nucleotide sequences of the nucleotide sequence represented by SEQ ID Nos: 1, 3 or 5 and labelling the polynucleotide with radioisotope markers or fluorescent markers according to the known methods using, for example, Random Primed DNA Labelling Kit (Boehringer Co.). Random Primer DNA Labelling Kit Ver. 2 (Takara Shuzo Co., Ltd.), ECL Direct Nucleic Acid Labelling and Detection System (Amersham Pharmacia Co.), Megaprime DNA-labelling system (Amersham Pharmacia Co.) and the like.

Examples of the hybridization conditions include stringent conditions and the following conditions may be exemplified: keeping at 65° C. in the presence of 6×SSC (0.9 M NaCl and 0.09 M trisodium citrate), 5× Denhard's solution [0.1% (w/v) ficoll 400, 0.1% (w/v) polyvinylpyrrolidone, 0.1% BSA], 0.5% (w/v) SDS and 100 μg/ml degenerated salmon sperm DNA or in DIG EASY Hyb solution (Boeringer-Mannheim Co.) containing 100 μg/ml of degenerated salmon sperm DNA, successively keeping at a room temperature for 15 minutes two times in the presence of 1×SSC (0.15 M NaCl and 0.015 M trisodium citrate) and 0.5% (w/v) SDS, and further keeping at 68° C. for 30 minutes in the presence of 0.1×SSC (0.015 M NaCl and 0.0015 M trisodium citrate) and 0.5% SDS.

In order to obtain a polynucleotide coding the cytokinin receptor of Arabidopsis, PCR can be carried out employing TAKARA LA tag™ (thermostable DNA polymerase) (Takara Shuzo Co., Ltd.) and using a solution containing cDNA library phage of Arabidopsis (about 1,000,000 pfu) as a template and a polynucleotide having the nucleotide sequence represented by SEQ ID No: 25 and a polynucleotide having the nucleotide sequence represented by SEQ ID No: 26 as a primer set to amplify and obtain DNA to be a probe. In order to obtain a polynucleotide coding the cytokinin biosynthesis enzyme of Arabidopsis, PCR can be carried out employing TAKARA LA taq™ (thermostable DNA polymerase) (Takara Shuzo Co., Ltd.) and using a solution containing cDNA library phage of Arabldopsis (about 1,000,000 pfu) as a template and polynucleotides having partial nucleotide sequence of the nucleotide sequence of SEQ ID Nos: 7, 9, 11, 13, 15, 17 or 19 as a primer set to amplify and obtain DNA to be a probe. The PCR solution to be used may be prepared by adding to 250 ng of cDNA library, the reaction solutions instructed by the kit.

The PCR conditions may be as follows: keeping at 94° C. for 2 minutes, and then at 8° C. for 3 minutes, and further repeating 40 cycles each of which comprising keeping at 94° C. for 30 seconds, at 55° C. for 30 seconds, and at 68° C. for 5 minutes.

Using the amplified and obtained DNA as a template, a ³²P-labeled probe may be produced employing Megaprime DNA-labelling system kit (Amersham Pharmacia Co.) and using the reaction solutions instructed by the kit. Using the probe obtained in such a manner, colony hybridization is carried out by a conventional method, practically the hybridization is carried out keeping at 65° C. in the presence of 6×SSC (0.9 M NaCl and 0.09 M trisodium citrate), 5× Denhard's solution [0.1% (w/v) ficoll 400, 0.1% (w/v) polyvinylpyrrolidone, 0.1% BSA], 0.5% (w/v) SDS and 100 μg/ml degenerated salmon sperm DNA or in the DIG EASY Hyb solution (Boeringer-Mannheim Co.) containing 100 μg/ml of degenerated salmon sperm DNA, successively keeping at a room temperature for 15 minutes two times in the presence of 1×SSC (0.15 M NaCl and 0.015 M trisodium citrate) and 0.5% (w/v) SDS, and further keeping at 68° C. for 30 minutes in the presence of 0.1×SSC (0.015 M NaCl and 0.0015 M sodium citrate) and 0.5% SDS to obtain clone hybridized with the probe.

Further, a polynucleotide coding the cytokinin receptor may be prepared based on, for example, the nucleotide sequence represented by SEQ ID No: 1, 3 or 5 by chemical synthesis of polynucleotides according to a conventional method such as phosphite-triester method (Hunkapiller, M. et al., Nature, 310, 105, 1984). In a similar manner, a polynucleotide coding the cytokinin biosynthesis enzyme may be prepared based on, for example, the nucleotide sequence represented by SEQ ID No: 7, 9, 11, 13, 15, 17 or 19 by chemical synthesis of polynucleotides.

The polynucleotide comprising a nucleotide sequence encoding an amino acid sequence of the cytokinin receptor and the polynucleotide comprising a nucleotide sequence encoding an amino acid sequence of a cytokinin biosynthesis enzyme, obtained in such a manner, may be cloned in a vector by a conventional method described in, “Molecular Cloning: A Laboratory Manual 2nd edition” (1989), Cold Spring Harbor Laboratory Press; “Current Protocols In Molecular Biology” (1987), John Wiley & Sons, Inc. ISBNO-471-50338-X, and the like. The vector to be used maybe, for example, pBlue Script II vector (produced by Stratagene Co.), pUC18/19 vector (produced by Takara Shuzo Co., Ltd.), TA cloning vector (produced by Invitrogen Co.), and the like

Incidentally, the nucleotide sequence of cloned polynucleotide may be confirmed by, the Maxam Gilbert method (described in, for example, Maxam, A., M & W. Gilbert, Proc. Natl. Acad. Sci. USA, 74, 560, 1977, and the like), the Sanger method (described in, for example, Sanger, F. & A. R. Coulson, J. Mol. Biol., 94, 441, 1975, Sanger, F. & Nicklen and A. R. Coulson., Proc. Natl. Acd. Sci. USA, 74, 5463, 1977, and the like). For the process, the following commercialized kits may be, for example, employed: Thermo Sequenase II dye terminator cycle sequencing kit (produced by Amersham Pharmacia Co.), BigDye Terminator Cycle Sequencing FS Ready Reaction Kit (produced by PE Biosystems Japan Co.), and the like.

(3) Construction of Expression Vector

An expression vector of a polynucleotide comprising a nucleotide sequence encoding an amino acid sequence of the cytokinin receptor and an expression vector of a polynucleotide comprising a nucleotide sequence encoding an amino acid sequence of the cytokinin biosynthesis enzyme may be constructed according to a conventional method described in, for example, Molecular Cloning 2nd edition written by J., Sambrook, E., F., Frisch, & T., Maniastis, published by Cold Spring. Harbor Laboratory Press.

Usable are vectors to be used in host cells to be transformed, for example, independently replicating vectors which contain genetic information possible to be duplicated in the host cells and further are possible to be isolated from the host cells and purified and may have detectable marker. More practically, in the case of using bacteria such as E. coli as the host cells, for example, Plasmid pUC119 (produced by Takara Shuzo Co., Ltd.), Phagemid pBluescript II (Stratagene Co.) and the like may be used. In the case of using yeast as the host cells, for example, Plasmid pACT2 (Clontech Co.) and the like may be used. In the case of using plant cells as the host cells, for example, a polynucleotide comprising a nucleotide sequence encoding an amino acid sequence of the cytokinin receptor or a polynucleotide comprising a nucleotide sequence encoding an amino acid sequence of the cytokinin biosynthesis enzyme may be integrated with plasmid pBI221 (Clontech Co.) to construct the vectors.

An expression vector possible to express a polynucleotide comprising a nucleotide sequence encoding an amino acid sequence of the cytokinin receptor in a host cell may be constructed by integrating a promoter with the aforementioned vectors in the upstream of the polynucleotide comprising a nucleotide sequence encoding an amino acid sequence of the cytokinin receptor in a binding manner of enabling to function in the host cell. In a similar manner, an expression vector possible to express a polynucleotide comprising a nucleotide sequence encoding an amino acid sequence of the cytokinin biosynthesis enzyme in a host cell may be constructed by integrating a promoter with the aforementioned vectors in the upstream of the polynucleotide comprising a nucleotide sequence encoding an amino acid sequence of the cytokinin biosynthesis enzyme in a binding manner of enabling to function in the host cell. In these cases, the phrase, “in a binding manner of enabling to function”, means that the polynucleotide comprising a nucleotide sequence encoding an amino acid sequence of the cytokinin receptor or the polynucleotide comprising a nucleotide sequence encoding an amino acid sequence of the cytokinin biosynthesis enzyme, and the promoter are bonded as to express the polynucleotide comprising a nucleotide sequence encoding an amino acid sequence of the cytokinin receptor or the polynucleotide comprising a nucleotide sequence encoding an amino acid sequence of the cytokinin biosynthesis enzyme in the host cell under the control of the promoter. Usable as the promoter possible to function in the host cell in the case of using E. coli as the host cell are, for example, a promoter (lacP) of lactose operon of E. coli, a promoter (trpP) of triptophan operon, a promoter (argP) of arginine operon, a promoter (galP) of galactose operon, a tac-promoter, T7-promoter, T3-promoter, λ-phage promoter, (λ-pL, λ-pR) and the like. In the case of using yeast as the host cell, it may be prepared by a conventional genetic engineering method [described in Method in Enzymology 101 part (p.192-201) by Ammerer, et. al.] from ADH1 promoter (the ADH1 promoter is available from the yeast expression vector pAAH5 which contains the ADH1 promoter and its terminator and which may be obtained from Washington Research Foundation). The ADH1 promoter is included in U.S. patent application No. 299,733 of Washington Research Foundation and in the case that it is used for industrial and commercial purposes in USA., it is required to obtain permission from the patent holder. In the case of using a plant cell as the host cell, usable examples are a nopaline synthesis enzyme gene (NOS) promoter, an octopine synthesis enzyme gene (OCT) promoter, a cauliflower mosaic virus (CaMV)-derived 19S promoter, a CaMV-derived 35S promoter and the like.

Further, in the case of integrating a polynucleotide comprising a nucleotide sequence encoding an amino acid sequence of the cytokinin receptor or a polynucleotide comprising a nucleotide sequence encoding an amino acid sequence of the cytokinin biosynthesis enzyme into a vector already having a promoter possible to function in a host cell, the polynucleotide comprising a nucleotide sequence encoding an amino acid sequence of the cytokinin receptor or the polynucleotide comprising a nucleotide sequence encoding an amino acid sequence of the cytokinin biosynthesis enzyme is inserted in the downstream of the promoter and the polynucleotide comprising a nucleotide sequence encoding an amino acid sequence of the cytokinin receptor or the polynucleotide comprising a nucleotide sequence encoding an amino acid sequence of the cytokinin biosynthesis enzyme is inserted in a manner of enabling to function. For example, the aforementioned plasmid pACT 2 for yeast comprises the ADH1 promoter and therefore, an expression vector possible to express the polynucleotide comprising a nucleotide sequence encoding an amino acid sequence of the cytokinin receptor or the polynucleotide comprising a nucleotide sequence encoding an amino acid sequence of the cytokinin biosynthesis enzyme in yeast, such as CG1945 (Clontech Co.) may be constructed by inserting the polynucleotide comprising a nucleotide sequence encoding an amino acid sequence of the cytokinin receptor or the polynucleotide comprising a nucleotide sequence encoding an amino acid sequence of the cytokinin biosynthesis enzyme in the downstream of the ADH1 promoter of the plasmid pACT2.

(4) Production of a Transformed Cell

A transformed cell to be used in the present invention may be produced by introducing the constructed expression vector into a host cell by a conventional method. As the host cell to be used for production of the transformed cell, examples are bacteria, yeast, plant cell and the like. As bacteria, examples are bacteria belonging to E. coli, Serratia, Bacillus, Brevibacterium, Corynebacterium, Microbacterium and the like. As yeast, examples are budding yeast and fission yeast. More particularly, examples are yeast belonging to Saccharomyces, Schizosaccharomyces and the like. As plant cell, examples are BY-2 strain, which is a cultured cell of tobacco, and BMS strain, which is a cultured cell of corn (Black Mexican Sweet), and the like.

As the method for introducing the expression vector into the aforementioned host cell, conventional introduction methods are employed according to the host cell to be transformed. For example, in the case of using bacteria as the host cell, the aforementioned expression vector may be introduced into a host cell by employing a conventional introduction method such as a calcium chloride method and an electroporation method described in, “Molecular Cloning” (by J. Sambrook, et. al Cold Spring Harbor 1989). In the case of using yeast as a host cell, the aforementioned expression vector may be introduced into the host cell by employing Yeast transformation kit (produced by Clontech Co.) based on a lithium method. Further, in the case of using a plant cell as a host cell, the aforementioned expression vector may be introduced into the host cell by a conventional introduction method, for example, Agrobacterium infection method (Japanese Examined Patent Application No. 2-58917, Japanese Laid-Open Patent Application No. 60-70080), an electroporation method into a protoplast (Japanese Laid-Open Patent Application No. 60-251887, Japanese Laid-Open Patent Application No. 5-68575), a particle gun method (Japanese Laid-Open Patent Application No.6-508316, Japanese Laid-Open Patent Application No. 63-258525).

A transformed cell in which a cytokinin receptor of partially transmembrane region-deleted type, i.e. a cytokinin receptor of a transmembrane quantity variation type is introduced, will be described below.

A cytokinin receptor to be used in the present invention includes a cytokinin receptor wherein said cytokinin receptor has at least one transmembrane region but less than that in its natural form (commonly 2 to 4 transmembrane region) (incidentally, in the present invention, such cytokinin receptors are sometimes referred as to cytokinin receptors of partially transmembrane region-deleted type.) In this case, the phrase, “its natural form” means cytokinin receptors having an amino acid sequence most frequently existing among organisms having the similar nomenclature and generally called also as wild-type cytokinin receptor.

Such cytokinin receptors of partially transmembrane regions-deleted type are cytokinin receptors whose transmembrane region structure may be determined by employing structure assumption software available in http://www.ch.embnet.org/software/TMPRED_form.html and whose transmembrane regions are partially deleted, for example, in 1 to 2 sites, and are less in number than the number of the transmembrane regions of the natural type cytokinin receptors (i.e. natural form).

More particularly, examples of such cytokinin receptors include a cytokinin receptor having the amino acid sequence from amino acid number 196 to 1176 among the amino acid sequence represented by SEQ ID No: 4 (2 transmembrane regions); a cytokinin receptor having the amino acid sequence from amino acid number 50 to 1176 among the amino acid sequence represented by SEQ ID No: 4 (3 transmembrane regions); a cytokinin receptor having the amino acid sequence from amino acid number 32 to 1036 among the amino acid sequence represented by SEQ ID No: 6 (2 transmembrane regions); a cytokinin receptor having the amino acid sequence derived from the amino acid sequences of these cytokinin receptors wherein one or a plurality of amino acids are deleted, substituted or added, for example, cytokinin receptors having the amino acid sequence derived from the amino acid sequences in which one methionine is added to the amino-terminal and the like.

The DNA coding the cytokinin receptor may be constructed as to hold transmembrane regions in a less number than the number of the transmembrane regions of the natural type cytokinin receptors by partially deleting the transmembrane regions by a conventional genetic engineering technique.

A transformed cell in which the polynucleotide comprising a nucleotide sequence encoding an amino acid sequence of the partially transmembrane region-deleted type cytokinin receptor and the polynucleotide comprising a nucleotide sequence encoding an amino acid sequence of the cytokinin biosynthesis enzyme are introduced may be produced according to the aforementioned method, “Production of a cell that is transformed with a polynucleotide comprising a nucleotide sequence encoding an amino acid sequence of a cytokinin receptor and a polynucleotide comprising a nucleotide sequence encoding an amino acid sequence of a cytokinin biosynthesis enzyme”.

A transformed cell to express chimera-type cytokinin receptor will be described below.

A cytokinin receptor to be used in the present invention also includes a chimera-type cytokinin receptor comprising an extracellular region of the cytokinin receptor, transmembrane regions of the cytokinin receptor, a histidine kinase region of the cytokinin receptor, and a receiver region for a histidine kinase. The chimera-type cytokinin receptor has the extracellular region, the transmembrane regions, and the histidine kinase region homogeneous to one another and the receiver region for the histidine kinase heterogeneous to these regions. For example, the chimera-type cytokinin receptor may have said extracellular region, transmembrane regions, and histidine kinase region derived from a cytokinin receptor selected from CRE1, AHK2, and AHK3, and have the receiver region derived from the osmosensor SLN1 in budding yeast.

The histidine kinase protein has the following sequence in common in plants, e.g. higher plants, and microorganism. That is, histidine kinase protein is composed of an extracellular region, transmembrane region (generally about 2 to 4), histidine kinase region having histidine kinase activity and holding Histidine residue to be an active site, and receiver region having a reception part for a phosphoryl group transfer and holding Aspartic acid residue to bean active site. In the chimera-type cytokinin receptor, it is preferable that the extracellular region, the transmembrane regions, and the histidine kinase region are all derived from the identical type of cytokinin receptor, whereas the receiver regions are derived from a histidine kinase protein that is different from type of the former cytokinin receptor.

It is sufficient for the receiver region of the chimera-type cytokinin receptor to have a function of receiving signals transmitted from the histidine kinase region and transmitting them to the next step and any of receiver region may be usable as long as they can complement or improve the intrinsic functions of the receiver region of a histidine kinase protein comprising the homogeneously derived extracellular region, transmembrane regions, and histidine kinase region.

As such a receiver region, usable are, for example, a receiver region of histidine kinase proteins derived from microorganism (e.g. a receiver region of histidine kinase protein derived from a microorganism such as yeast, E. coli) and more particularly receiver region of histidine kinase protein coded in SLN1 gene derived from budding yeast (e.g. the amino acid sequence represented by SEQ ID No: 21), receiver region of histidine kinase protein coded in Chey gene derived from Salmonella, receiver region of histidine kinase protein coded in RcsC gene, which is a hybrid sensor of E. coli [Maeda T, et al. Nature: 369 242-245, (1994): e.g. the amino acid sequence represented by SEQ ID No: 22], receiver region of histidine kinase protein coded in Phks gene relevant to cell cycle control of fission yeast [Shieh, J C, et al., Gene Dev. 11, 1008-1022(.1997)].

In the above, histidine kinase regions of the cytokinin receptor may include the region present C-terminally downstream from the particular transmembrane region which is the most C-terminally downstream transmembrane region of the cytokinin receptor. Said histidine kinase region typically has five conserved motifs which are common to generic histidine kinases as described in Annual Review of Genetics 23:311-336 (1989), Microbiological Reviews 53(4):450-490(1989), Science 262:539-544(1993), and the like. Examples of the region includes a region having the amino acid sequence from amino acid number 587 to 844 among the amino acid sequence represented by SEQ ID No: 4 in a case of AHK2, a region having the amino acid sequence from amino acid number 450 to 700 among the amino acid sequence represented by SEQ ID No: 6 in a case of AHK3 and a region having the amino acid sequence from amino acid number 449 to 714 among the amino acid sequence represented by SEQ ID No: 2 in a case of CRE1.

Receiver regions of the cytokinin receptor may include the region existing between the histidine kinase region and the C-terminal end of the cytokinin receptor. Said region having three conserved motifs which are common to generic histidine kinases as described in Annual Review of Genetics 23:311-336 (1989), Science 262:539-544(1993), and the like. Examples of the region includes a region having the amino acid sequence from amino acid number 891 to 1163 among the amino acid sequence represented by SEQ ID No: 4 in a case of AHK2, a region having the amino acid sequence from amino acid number 746 to 1018 among the amino acid sequence represented by SEQ ID No: 6 in a case of AHK3 and a region having the amino acid sequence from amino acid number 763 to 1038 among the amino acid sequence represented by SEQ ID No: 2 in a case of CRE1.

In addition, a sensor region for cytokinin means, for example, a region which is a part of any of the extracellular regions of the cytokinin receptor, wherein said sensor region is present between a transmembrane region next to the histidine kinase region and a transmembrane region secondary close to the histidine kinase region. Said sensor region typically has 50% or more identity and homology between three cytokinin receptors of AHK2, AHK3 and CRE1 as described in Plant and Cell Physiology 42(2):231-235(2001) and the like. Examples of the region includes a region having the amino acid sequence from amino acid number 259 to 536 among the amino acid sequence represented by SEQ ID No: 4 in a case of AHK2, a region having the amino acid sequence from amino acid number 120 to 399 among the amino acid sequence represented by SEQ ID No: 6 in a case of AHK3 and a region having the amino acid sequence from amino acid number 132 to 398 among the amino acid sequence represented by SEQ ID No: 2 in a case of CRE1.

A polynucleotide comprising a nucleotide sequence encoding an amino acid sequence of the chimera-type cytokinin receptor may be constructed by respectively producing polynucleotides encoding each of the extracellular region of the cytokinin receptor, transmembrane regions of the cytokinin receptor, histidine kinase region of the cytokinin receptor, and receiver region for the histidine kinase protein, joining the polynucleotides by a conventional genetic engineering technique so as to prevent appearance of any termination codon in the middle while using a proper linker so as to prevent frame shift. Incidentally, one polynucleotide may be utilized to provide the polynucleotide encoding the extracellular region of the cytokinin receptor and transmembrane regions of the cytokinin receptor, or the polynucleotide encoding the extracellular region of the cytokinin receptor, transmembrane regions of the cytokinin receptor, and histidine kinase region of the cytokinin receptor.

The polynucleotides coding amino acid sequences of the aforementioned respective regions may respectively be produced by known methods. For example, in the case of production by PCR, at first, the oligonucleotides (5′ side primers) having the nucleotide sequences of 5′ terminal regions of respective regions to be amplified and oligonucleotides (3′ side primers) having complementary nucleotide sequences to the nucleotide sequences of 3′ terminal are designed and synthesized. The primers may be oligonucleotides of about 14 nucleotides to about 35 nucleotides in general and are preferable to contain in the 5′ side of the primers, restriction enzyme recognition sequences usable at the time of ligating the polynucleotides amplified by the PCR to one another or these polynucleotides to vectors. Then, using the primers and the cDNA library as a template, amplification reactions may be carried out in the conventional reaction conditions employed for the PCR. As the template to be used in the case of producing the polynucleotide coding amino acid sequence of the extracellular region, the transmembrane regions of the cytokinin receptor or the histidine kinase region of the cytokinin receptor, usable is cDNA library derived from plants such as higher plants. Also as the template to be used in the case of producing the polynucleotides coding amino acid sequence of the receiver regions of the histidine kinase, usable is the total DNA or cDNA library derived from microorganism prepared by a conventional method.

The transformed cell in which the polynucleotide comprising a nucleotide sequence encoding an amino acid sequence of the chimera-type cytokinin receptor and the polynucleotide comprising a nucleotide sequence encoding an amino acid sequence of the cytokinin biosynthesis enzyme are introduced may be produced according to the aforementioned, “Production of a cell that is transformed with a polynucleotide comprising a nucleotide sequence encoding an amino acid sequence of a cytokinin receptor and a polynucleotide comprising a nucleotide sequence encoding an amino acid sequence of a cytokinin biosynthesis enzyme”.

Intracellular signal transduction system relevant to cytokinin will be described below.

Measurement of the existence or the quantity of intracellular signal transduction from the cytokinin receptor expressed in the transformed cell produced by the above-described manner may be carried out by utilizing the intracellular signal transduction system originally present in the host cell used for production of the transformed cell. The existence or the quantity of intracellular signal transduction can be determined by employing the quantity of the cell growth of the transformed cell as an indicator. Alternatively, a regulator (and/or a mediator), which is a downstream signal transducer in the so-called Two-Component regulatory system, can be additionally introduced and expressed in the host cell and the expressed system may be used in the intracellular signal transduction system. Usable as the Two-Component regulatory system, for example, are Two-Component regulatory systems corresponding to 5 types of ethylene receptors; ETR1, ETR2, ERS1, ERS2, and EIN4; present in Arabidopsis [Chang et al., Science 262: 539-544 (1993), Hua et al., Science 269: 1712-1714 (1995), Sakai et al., Plant Cell Physiol 39: 1232-1239 (1998)] and AtHK1 having sensor functions to the osmotic pressure [Urao, Plant ell 11: 1743-1754 (1999)].

As the host cell to be used for production of such a transformed cell, usable are host cells improved as to have histidine kinase activity lower than the intrinsic histidine kinase activity of the host cells. For example, it includes the host cells improved as to have histidine kinase activity lower than the intrinsic histidine kinase activity of the host cell by deleting one or more of histidine kinases. The phrase, “histidine kinase activity lower than” means that the quantity of a phosphoryl group transfer from Histidine residue (the active site of histidine kinase regions having histidine kinase activity) to Aspartic acid residue (the active site of receiver regions having a reception part) is decreased. Such improvements in the host cell may provide a change of the quantity of the cell growth, change of the morphology, change of the shape, change of the quantity of the biosynthesis of specific compound, change of the quantity of the metabolism of specific compound in the transformed cell. More particularly, the following strain (Maeda T et al., Nature 369: 242-245 (1994)] may be exemplified: a strain obtained by defecting the SLN1 gene coding the protein having the osmotic pressure sensor function and derived from the budding yeast such as Saccharomyces cerevisiae and the like. Since the strain is defected in the histidine kinase protein existing in Saccharomyces cerevisiae, there can be more clearly detected the existence or the quantity of the intracellular signal transduction from the cytokinin receptor expressed in the transformed cell by using the quantity of the cell growth of the transformed cell as an indicator. Further, other preferable example includes an E. coli strain defective in the RcsC gene that encodes a hybrid sensor, and a fission yeast strain defective in Phks gene that is relevant to the cell cycle.

Method for analyzing the activity of controlling biosynthesis of cytokinin will be described below.

In the method for analyzing the activity of controlling biosynthesis of cytokinin, examples of the first step of bringing an examinee substance into contact with a transformed cell into which a polynucleotide comprising a nucleotide sequence encoding an amino acid sequence of a cytokinin receptor and a polynucleotide comprising a nucleotide sequence encoding an amino acid sequence of a cytokinin biosynthesis enzyme are introduced include a method for culturing the transformed cell in a culture medium containing the examinee substance. In order to culture the transformed cell, the following cultures are usable: liquid-phase culture for culturing the transformed cell in a liquid culture medium and a solid-phase culture for culturing the transformed cell in a solid culture medium produced by adding agar or the like to the liquid culture medium. The concentration of an examinee substance in the culture medium is about 1 nM to about 1 mM and preferably about 10 nM to about 100 μM. The culture time is, for example, 1 hour to 3 days and preferably 25 hours to 2 days.

Following the above-described first step, the existence or the quantity of intracellular signal transduction from the cytokinin receptor expressed in the transformed cell may be measured. Using the measured value as an indicator, the activity of the examinee substance to control biosynthesis of cytokinin may be analyzed.

More particularly, for example, in the case of using a transformed cell of the present invention (that is, a transformed cell, wherein growth of said cell is directly controlled by intracellular signal transduction from the cytokinin receptor) produced using TM1B2 (sln1 Δ) [Maeda T et al, Nature 369: 242-245 (1994)], a SLN1 genetically defected strain in which PTP2 Tyrosine phosphatase gene [Ota et al. Proc. N. A. Sci., USA, 89, 2355-2359 (1992)] is introduced, as a host cell, the activity of controlling biosynthesis of cytokinin may be measured using, as an indicator, the quantity of the cell growth of the transformed cell in a culture medium (an agar culture medium or a liquid culture medium) containing glucose as a carbon source, for example, a DOHLU+Glu culture medium. In the case of the DOHLU +Glu culture medium to which the examinee substance is added, the examinee substance found capable of changing growth of the transformed cell can be evaluated as a substance having the activity of controlling biosynthesis of cytokinin, as compared with growth of the transformed cell in the DOHLU+Glu culture medium to which the examines substance is not added (a culture medium containing no substance having the activity of controlling biosynthesis of cytokinin). Incidentally, as a control to verify that the change of cell growth of the transformed cell is not resulted from activity of the examinee substance directly controlling signal transduction from cytokinin receptor, such as agonist-activity or antagonist-activity to the cytokinin receptor, TM182 (sln1 Δ) transformed with only a polynucleotide comprising a nucleotide sequence encoding an amino acid sequence of a cytokinin receptor may be cultured in DOLU+Glu culture medium with or without the examinee substance. In a case where the examinee substance dose not affect growth of the control cell, the examinee substance may not directly affect signal transduction system from cytokinin receptor. As a control to verify that the change of cell growth of the transformed cell is not resulted from activity of the examinee substance which affects somewhere not involved in biosynthesis of cytokinin nor signal transduction from cytokinin receptor, TM182 (sln1 Δ) may be cultured in culture medium containing galactose as the carbon source in place of glucose, such as DOLU+Gal culture medium with or without the examinee substance. In a case where the examinee substance dose not affect growth of the control cell [TM182 (sn1 Δ)], the examinee substance may not affect somewhere not involved in biosynthesis of cytokinin nor signal transduction from cytokinin receptor.

Further, in the case of using a transformed cell of the present invention (that is, a transformed cell, wherein growth of said cell is directly controlled by the intracellular signal transduction from the cytokinin receptor) produced by employing fission yeast as a host cell, such as a Phks genetically defected strain, the fission pattern of the fission yeast may be observed with a microscope. In this case, when the examinee substance is added to the culture medium, the examinee substance found capable of changing fission and propagation of the transformed cell can be evaluated as a substance having the activity of controlling biosynthesis of cytokinin, as compared with fission and propagation of the transformed cell in a medium to which the examinee substance is not added.

Furthermore, in the case of using a transformed cell of the present invention produced by employing as the host cell an E. coli defective in RcsC gene in to which cps-LacZ is introduced, the X-Gal coloring may be observed in an agar culture medium or a liquid culture medium [Suzuki et al. Plant Cell Physiol 42: 107-113 (2001)]. In this case, when the culture medium contains the examinee substance, the examinee substance found capable of changing coloring of the transformed cell can be evaluated as a substance having the activity of controlling biosynthesis of cytokinin, as compared with coloring of the transformed cell in a medium to which the examinee substance is not added.

The activity of the examinee substance for controlling biosynthesis of cytokinin can be detected by evaluating the activity of two or more examinee substances. The activity of controlling biosynthesis of cytokinin can be provided for the evaluation by conducting the aforementioned method for analyzing the activity of controlling biosynthesis of cytokinin. For example, the evaluation of the activity of controlling biosynthesis of cytokinin can be based on the difference between the two or more examinee substances, which are obtained by comparison of the existence or the quantity of the intracellular signal transduction. In utilizing the two or more different examinee substances, the examinee substances are typically utilized independently in different systems. It is preferable that at least one of the two or more examinee substances has no activity of controlling biosynthesis of cytokinin.

Moreover, a substance with the activity of controlling biosynthesis of cytokinin may be searched by selecting substances having the activity of controlling biosynthesis of cytokinin based on the activity evaluated by the aforementioned detection methods.

Further, a substance selected by the above described detection methods may be utilized as an active ingredient of a plant growth regulator.

The plants to be the objects to be treated with the aforementioned plant growth regulator are, for example, decorative plants such as flowering plants and ornamental foliage plants; cultivating plants such as crop, vegetable, fruit and the like; fibrous plants; trees; lawn and the like.

The growth regulator is generally mixed with a solid carrier, a liquid carrier, and the like and further, if needed, mixed with a surfactant and other auxiliary agents for the formulation of an agricultural and horticultural agent and formulated in an emulsion agent, a hydrating agent, a suspension agent, a solution agent and the like. In these agricultural and horticultural agents, an agonist-active substance or an antagonist-active substance to the cytokinin receptor may be contained generally in 0. 5 to 90% by weight and preferably in 1 to 80% by weight.

Usable as the solid carrier to be used for the formulation of an agricultural and horticultural agent are, for example, clays (kaolinite, kieselguhr, synthesized hydrated silicon oxide, intercalated clays, bentonite, acidic white clay, and the like), talc, other inorganic minerals (sericite, quartz powder, sulfur powder, activated carbon, calcium carbonate, and the like), chemical fertilizers (ammonium sulfate, ammonium phosphate, ammonium nitrate, ammonium chloride, urea, and the like) in finely powdered state or in granular state and usable as the liquid carrier are, for example, water, alcohols (methanol, ethanol and the like), ketones (acetone, methyl ethyl ketone, cylohexanone and the like), aromatic hydrocarbons (toluene, xylene, ethylbenzene, methylnaphthalene and the like), non-aromatic hydrocarbons (hexane, cyclohexane, kerosene and the like), esters (ethyl acetate, butyl acetate and the like), nitrites (actonitrile, isobutylnitrile and the like), ethers (dioxane, diisopropyl ether and the like), acid amides (dimethylformamide, dimethylacetamide and the like), halohydrocarbons (dichloroethane, trichloroethane and the like), etc.

As the surfactant, usable are, for example, alkylsulfuric acid esters, alkylsulfonic acid salts, alkylarylsulfonic acid salts, alkylaryl ethers and their polyoxyethylene compounds, polyethylene glycols, polyhydric alcohol esters, sugaralcohols and the like.

As other auxiliary agents for the formulation of agricultural and horticultural agents, usable are solidification agents and dispersanst such as casein, gelatin, polysaccharides (starch, acacia, cellulose derivatives, alginic acid and the like), lignin derivatives, bentonite, synthesized water-soluble polymer [poly(vinyl alcohol), poly(vinyl pyrolidone), poly(acrylic acid) and the like] and the like, and stabilizers such as PAP (acidic isopropyl phosphate), BHT (2,6-tert-butyl-4-methylphenol), BHA (2-/3-tert-butyl-4-methoxyphenol), plant oils, mineral oils, aliphatic acids, aliphatic acid esters and the like.

The substance with the activity of controlling biosynthesis of cytokinin made to be agricultural and horticultural agents is used as it is or diluted with water to carry out treatment for the stem and leave parts, branch and leave parts, and flower and fruit parts of plants by spraying, for fruits by immersion, and for fruits by application. The plant growth regulator is used for the object plants to carry out the treatment once or a plurality of times.

In the case of using the plant growth regulator for the purpose of suppressing the dropping of fruits, the plant growth regulator is diluted with water and the resulting diluted agent is sprayed to the fruit parts and branch and leave parts before harvest.

In the case of using the plant growth regulator for the purpose of suppressing the ball dropping of cotton, the plant growth regulator is diluted with water and the resulting diluted agent is sprayed to the balls and stem and leave parts of cotton before harvest.

The plant growth regulator may be used for the treatment of growing plants or of plants after harvest.

The appilcation amount of the substance with the activity of controlling biosynthesis of cytokinin in an agricultural and horticultural agent is generally 1 to 8000 g per 1 hectare, although it is changed depending on the state of the agricultural and horticultural agent, the timing for the treatment, the method for the treatment, the site for the treatment, and the object plant to be treated. Also in the case of using the plant growth regulator while diluting the agent with water, the concentration of the agent is generally 0.0001 to 1000 mM and preferably 0.001 to 10 mM, although it is changed depending on the state of the agricultural and horticultural agent, the timing for the treatment, the method for the treatment, the site for the treatment, and the object plant to be treated.

Next, hereinafter given are formulation examples of an agricultural and horticultural agent produced from a substance with the activity of controlling biosynthesis of cytokinin and used as a plant growth regulator. The parts in the following description of the examples denotes the parts by weight.

### FORMULATION EXAMPLE 1

A hydrating agent was obtained by sufficiently pulverizing and mixing 50 parts of a substance with the activity of controlling biosynthesis of cytokinin, 3 parts of calcium ligninsulfonate, 2 parts of sodium laurylsulfate, and 45 parts of synthesized hydrated silicon oxide.

### FORMULATION EXAMPLE 2

A hydrating agent was obtained by sufficiently pulverizing and mixing 70 parts of a substance with the activity of controlling biosynthesis of cytokinin, 3 parts of calcium ligninsulfonate, 2 parts of sodium laurylsulfate, and 25 parts of synthesized hydrated silicon oxide.

### FORMULATION EXAMPLE 3

An emulsion agent was obtained by mixing 40 parts of a substance with the activity of controlling biosynthesis of cytokinin, 3 parts of polyoxyethylene sorbitane monoolate, 2 parts of CMC (carboxymethyl cellulose), and 52 parts of water and wet-pulverizing the resultant mixture to be 5 μm or smaller in the particle size.

The present invention makes it possible to provide a transformed cell co-expressing a cytokinin receptor and a cytokinin biosynthesis enzyme. Further, by using such a transformed cell, it is possible to provide a method for analyzing the activity of controlling biosynthesis of cytokinins. Furthermore, by using such an analysis method, it is possible to provide a method for quickly finding substances having the activity of controlling biosynthesis of cytokinins with small amounts of samples.

### EXAMPLES

Hereinafter, although the present invention will be described in details with the reference to examples, the present invention is not at all restricted to these examples.

### Example 1

### Preparation of a Polynucleotide Comprising a Nucleotide Sequence Encoding an Amino Acid Sequence of a Cytokinin Receptor and Preparation of an Expression Vector of the Polynucleotide (Part 1): Production of Arabidopsis cDNA Phage Library for CRE1 Cloning

Seeds of Arabidopsis thaliana ecotype Wassilewskija were sterilized with 70% of ethyl alcohol for 1 minute and were further sterilized with 1.5% of sodium hypochlorlte for 10 minutes. The resulting seeds were well washed with sterilized water and then were cultured for 2 weeks in GM culture medium [4.3 g Murashige and Skoog's basal salt mixture, 1% sucrose, 10 ml of 5% MES-KOH (pH 5.7), 0.3% PHYTAGEL™ (gellan gum)(SIGMA)] to obtain 5 g of the plant. After the plant was frozen in liquified nitrogen and physically milled with a mortar and a pestle. The resulting milled product was mixed with a mixed solution of 10 ml of an extraction buffer [200 mM Tris-HCl (pH 8.5). 100 mM NaCl, 10 mM EDTA, 0.5% SDS, 14 mM 8-mercaptoethanol] and 10 g of phenol. After being mixed by a Vortex mixer, the resulting mixture was mixed further with 10 ml of chloroform and vigorously stirred and subjected to centrifugal separation at 10,000 rotation for 20 minutes. The recovered aqueous layer was mixed with LiCl in the concentration to be 2M of the final concentration, left still at −80° C. for 3 hours, thawed and subjected to centrifugal separation at 10,000 rotation for 20 minutes to recover a precipitate. The recovered precipitate was dissolved in 2 ml of TE [10 mM Tris-HCl (pH 8.0), 1 mM EDTA] and then further mixed with 0.2 ml of 3 M sodium acetate (pH 5.2) and. 5 ml ethanol and subjected to centrifugal separation to recover RNA as a precipitate. Further, the precipitate (RNA) was subjected to treatment with OLIGOTEX™ dT30 super (oligo d(T) latex beads).(Nippon Rosch Co.) to extract RNA integrated with polyA.

The production of phage cDNA library from the extracted RNA integrated with polyA was carried out employing ZAP-cDNAR Synthesis Kit (Stratagene Co.) according to the instruction. The potency of the produced phage cDNA library was 500,000 PFU.

### Example 2

### Preparation of a Polynucleotide Comprising a Nucleotide Sequence Encoding an Amino Acid Sequence of a Cytokinin Receptor and Preparation of an Expression Vector of the Polynucleotide (Part 1); Production of DNA Probe of CRE1

The PCR was carried out employing TAKARA LA TAQ™ (thermostable DNA polymerase) (Takara Shuzo Co., Ltd.) and using a phage solution (about 1,000,000 PFU) of the phage cDNA library produced in the example 1 as a template and a polynucleotide having the nucleotide sequence represented by SEQ ID No: 25 and a polynucleotide having the nucleotide sequence represented by SEQ ID No: 26 as the primers to amplify DNA. The procedure will be described in details below.

A PCR solution was prepared by adding a reaction composition containing dNTP and the like to the phage 1,000,000 pft and respective primer each in 0.2 μM according to the instruction of the kit and the desired DNA fragment was amplified in PCR conditions: keeping at 94° C. for 2 minutes, and further repeating 40 cycles each of which comprised keeping at 94° C. for 30 seconds, at 55° C. for 30 seconds, and at 68° C. for 5 minute. Next, using the amplified DNA fragment as a template, a probe labelled with ³²P was prepared employing Megaprime DNA-labelling system kit (Amersham Pharmacia Co.). Incidentally, reaction solution (25 μl) was prepared by adding 2.0 MBq of ³²P dCTP to 25 ng of amplified DNA fragment and adding a reaction composition instructed by the kit. The labelling reaction was carried out at 37° C. for 10 minutes.

### Example 3

### Preparation of a Polynucleotide Comprising a Nucleotide Sequence Encoding an Amino Acid Sequence of a Cytokinin Receptor and Preparation of an Expression Vector of the Polynucleotide (Part 1): Production of Phage cDNA Clone Holding CRE1 Gene

The cloning of desired CRE1 gene was carried out by plaque hybridization using the probe prepared by the example 2. Detailed description will be given below.

Using the phage cDNA library produced in the example 1 and according to the instruction of ZAP-cDNAR Synthesis Kit, plaque was produced. DNA was adsorbed on a nitrocellulose filter from the produced plague and then treated with UV rays to be fixed on the filter. Using the filter prepared in such a manner, hybridized phage cDNA clone was obtained by keeping at 65° C. in the presence of 6×SSC (0.9M NaCl and 0.09 M trisodium citrate), 5× Denhard's solution [0.1% (w/v) ficoll 400, 0.1% (w/v) polyvinylpyrrolidone, 0.1% BSA], 0.5% (w/v) SDS and 100 μg/ml degenerated salmon sperm DNA or in DIG EASY Hyb solution (Boeringer-Mannheim Co.) containing 100 μg/ml of degenerated salmon sperm DNA, successively keeping at a room temperature for 15 minutes two times in the presence of 1×SSC (0.15 M NaCl and 0.015 M trisodium citrate) and 0.5% (w/v) SDS, and further keeping at 68° C. for 30 minutes in the presence of 0.1×SSC (0.015 M NaCl and 0.0015 M trisodium citrate) and 0.5% SDS.

### Example 4

### Preparation of a Polynucleotide Comprising a Nucleotide Sequence Encoding an Amino Acid Sequence of a Cytokinin Receptor and Preparation of an Expression Vector of the Polynucleotide (Part 1): Cloning of CRE1 cDNA

Using the cDNA of the phage cDNA clone obtained by the example 3 as a template and a polynucleotide having the nucleotide sequence represented by SEQ ID No: 23 and a polynucleotide having the nucleotide sequence represented by SEQ ID No: 24 as primers, DNA having the nucleotide sequence represented by SEQ ID No: 1 was amplified by PCR. Detailed description will be given below.

The PCR was carried out employing Herculase Enhanced DNA Polymerase (Stratagene Co., Ltd.) in the reaction conditions of keeping at 94° C. for 1 minute, and further repeating 25 cycles each of which comprised keeping at 94° C. for 30 seconds, at 55° C. for 30 seconds, and at 72° C. for 4 minute. Incidentally, the PCR solution (50 μl) was prepared by adding a reaction composition containing dNTP and the like to 500 ng of cDNA of the phage cDNA clone and respective primer each in 100 ng according to the instruction of the kit.

The desired DNA fragment was amplified in such a manner.

### Example 5

### Preparation of a Polynucleotide Comprising a Nucleotide Sequence Encoding an Amino Acid Sequence of a Cytokinin Receptor and Preparation of an Expression Vector of the Polynucleotide (Part 1): Construction of CRE1 Expression Vector

After p415CYC1, a yeast expression vector, [Munberg et al. Gene: 156 119-122 (1995), available from ATCC library (No. 873821)] was digested with a restriction enzyme Sma I and then using T4 DNA ligase, DNA having the nucleotide sequence represented by SEQ ID No: 1 and obtained by the example 4 was ligated downstream of the CYC 1 promoter sequence of the expression vector p415CYC1 as to be integrated to express the desired protein in yeast. The constructed DNA was confirmed to be in the right direction and its nucleotide sequence was confirmed to be the nucleotide sequence represented by SEQ ID No: 1 by an automatic DNA sequencer and then the expression plasmid p415CYC-CRE 1 was obtained.

### Example 6

### Preparation of a Polynucleotide Comprising a Nucleotide Sequence Encoding an Amino Acid Sequence of a Cytokinin Receptor and Preparation of an Expression Vector of the Polynucleotide (Part 2): Cloning of AHK3 (AAF99730) cDNA

Seeds of Arabidopsis thaliana ecotype Wassilewskija were sterilized with 70% of ethyl alcohol for 1 minute and further sterilized with 1.5% of sodium hypochlorite for 10 minutes. The resulting seeds were well washed with sterilized water and then cultured for 2 weeks in GM culture medium [4.3 g Murashige and Skoog's basal salt mixture, 1% sucrose, 10 ml of 5% MES-KOH (pH 5.7), 0.3% PHYTAGEL™ (gellan gum)(SIGMA)] to obtain 5 g of the plant. After the plant was frozen in liquif ied nitrogen and physically milled with a mortar and a pestle. The resulting milled product was mixed with a mixed solution of 10 ml of an extraction buffer [200 mM Tris-HCl (pH 8.5), 100 mM NaCl, 10 mM EDTA, 0.5W SDS, 14 mM β-mercaptoethanol] and 10 g of phenol. After being mixed by a Voltex mixer, the resulting mixture was mixed further with 10 ml of chloroform and vigorously stirred and subjected to centrifugal separation at 10,000 rotation for 20 minutes. The recovered aqueous layer was mixed with LiCl in the concentration to be 2M of the final concentration, left still at −80° C. for 3 hours, thawed and subjected to centrifugal separation at 10,000 rotation for 20 minutes to recover a precipitate. The recovered precipitate was dissolved in 2 ml of TE [10 mM Tris-HCl (pH 8.0), 1 mM EDTA] and then further mixed with 0.2 ml of 3 M sodium acetate (pH 5.2) and 5 ml ethanol and subjected to centrifugal separation to recover RNA as a precipitate. Further, 40 μg of the precipitate (RNA) was mixed with 30 unit of FPLC pure™ DnaseI (RNase free-DNase I) (Amersham-Pharmacia) and 60 unit of Superace (Ambion) to remove mixed genome DNA and the resulting RNA was subjected to the phenol/chloroform treatment and ethanol treatment to purify the RNA. Next, using the purified RNA as a template and oligo (dT) 12-18 (Amersham-Pharmacia) as a primer, RT-PCR was carried out. The RT-PCR was carried out employing Superscript II (GIBCO BRL Co.) at 42° C. for 40 minutes. Incidentally the PT-PCR solution was prepared according to the method described in instruction of the Superscript II.

The desired cDNA was amplified in such a manner.

Using the amplified cDNA as a template and a polynucleotide having the nucleotide sequence represented by SEQ ID No: 27 and a polynucleotide having the nucleotide sequence represented by SEQ ID No: 28 as primers, DNA having the nucleotide sequence represented by SEQ ID No: 5 was amplified by PCR. The PCR was carried out using Herculase Enhanced DNA Polymerase (Stratagene Co., Ltd.) in the reaction conditions of keeping at 94° C. for 1 minute, and further repeating 41 cycles each of which comprised keeping at 94° C. for 30 seconds, at 55° C. for 30 seconds, and at 72° C. for 4 minute. Incidentally, the PCR solution (50 μl) was prepared by adding a reaction composition containing dNTP and the like to 500 ng of the template DNA and respective primer each in 100 ng according to the instruction of the kit.

The desired DNA fragment was amplified in such a manner.

### Example 7

### Preparation of a Polynucleotide Comprising a Nucleotide Sequence Encoding an Amino Acid Sequence of a Cytokinin Receptor and Preparation of an Expression Vector of the Polynucleotide (Part 2): Construction of pHM-1

After p415CYC1 [Munberg et al. Gene: 156 119-122 (1995); available from ATCC library (No. 87382)] was digested with the restriction enzyme Spe I and BamH I, synthesized DNA fragments (linkers) having the nucleotide sequence represented by SEQ ID Nos: 29 and 30 were inserted to the expression vector p415CYC1 using T4 DNA ligaseas to newly add the restriction enzyme sites Sac II, Apa I, Nhe I to the plasmid p415CYC1 and construct pHM-1.

### Example 8

### Preparation of a Polynucleotide Comprising a Nucleotide Sequence Encoding an Amino Acid Sequence of a Cytokinin Receptor and Preparation of an Expression Vector of the Polynucleotide (Part 2): Construction of AHK3 Expression Vector

After the pHM-1 was digested with the restriction enzyme Sal I and Sac II, and then using T4 DNA ligase, DNA having the nucleotide sequence represented by SEQ ID No: 5 was introduced downstream of the CYC1 promoter sequence of the expression vector pHM-1 as to be integrated to express the desired protein in yeast. The constructed DNA was confirmed to be right in the direction and its nucleotide sequence was confirmed to be right in the sequence by an automatic DNA sequencer and thus the expression plasmid p415CYC-AHK3 was obtained.

### Example 9

### Preparation of a Polynucleotide Comprising a Nucleotide Sequence Encoding an Amino Acid Sequence of a Cytokinin Receptor and Preparation of an Expression Vector of the Polynucleotide (Part 2): Cloning of AHK2(BAB09274) cDNA

Using the cDNA prepared in the example 6 (the cDNA prepared by reverse transcription of the total RNA) as a template and a polynucleotide having the nucleotide sequence represented by SEQ ID No: 31 and a polynucleotide having the nucleotide sequence represented by SEQ ID No: 32 as primers, DNA having the nucleotide sequence represented by SEQ ID No: 3 was amplified by PCR. The PCR was carried out employing TAKARA Pfu Turbo denature (Takara Shuzo Co., Ltd.) in the reaction conditions of keeping at 94° C. for 1 minute, and further repeating 30 cycles each of which comprised keeping at 94° C. for 30 seconds, at 55° C. for 30 seconds, and at 72° C. for 4 minute. Incidentally, the PCR solution (50 μl) was prepared by adding a reaction composition containing dNTP and the like to 500 ng of the template DNA and respective primer each in 50 ng according to the instruction of the kit. The desired DNA fragment was amplified in such a manner.

### Example 10

### Preparation of a Polynucleotide Comprising a Nucleotide Sequence Encoding an Amino Acid Sequence of a Cytokinin Receptor and Preparation of an Expression Vector of the Polynucleotide (Part 2): Cloning of AHK2(BAB09274)ΔcDNA

Using the cDNA prepared in example 6 (the cDNA prepared by reverse transcription of the total RNA) as a template and a polynucleotide having the nucleotide sequence represented by SEQ ID No: 33 and a polynucleotide having the nucleotide sequence represented by SEQ ID No: 34 as primers, PCR was carried out to amplify DNA containing the nucleotide sequence represented by nucleotide numbers 586 to 3531 of SEQ ID No: 3 in which ATG was added to the 5′ terminal sites of said nucleotide sequence. The PCR was carried out employing TAKARA Pfu Turbo denature (Takara Shuzo Co., Ltd.) in the reaction conditions of keeping at 94° C. for 1 minute, and further repeating 30 cycles each of which comprised keeping at 94° C. for 30 seconds, at 55° C. for 30 seconds, and at 72° C. for 4 minute. Incidentally, the PCR solution (50 μl) was prepared by adding a reaction composition containing dNTP and the like to 500 ng of the template DNA and respective primer each in 50 ng according to the instruction of the kit. The desired DNA fragment was amplified in such a manner.

### Example 11

### Preparation of a Polynucleotide Comprising a Nucleotide Sequence Encoding an Amino Acid Sequence of a Cytokinin Receptor and Preparation of an Expression Vector of the Polynucleotide (Part 2): Construction of AHK2 and AHK2 Δ Expression Vectors

After pHM-1 was digested with a restriction enzyme Sac II and Nhe I and then using T4 DNA ligase, DNA fragments obtained by the example 9 and the example 10 were respectively ligated downstream to the CYC1 promoter sequence of the expression vector pHM-1 as to be integrated to express the desired protein in yeast. The introduced DNA was confirmed to be right in the direction and its nucleotide sequence was confirmed to be right in the sequence by an automatic DNA sequencer and thus the expression plasmid p415CYC-AHK 2 and p415CYC-AHK2Δ were obtained.

### Reference Example 1

### Production of Transformed Cells TM182-CRE1, TM182-AHK2, TM182-AHK2Δ and TM182-AHK3

Transformation of TM182 (sln1Δ), SLN1 genetically defected strain, [Maeda T et al. Nature: 369 242-245 (1994)] was carried out using the obtained expression plasmid p415CYC-CRE1 (the example 5), p415CYC-AHK2 (the example 11), p415CYC-AHK2Δ (the example 11) and p415CYC-AHK3 (the example 8). The transformation was carried out by employing Polyethylene glycol/lithium acetate (PEG/LiAc)-mediated transformation method according to the description of VII. Library Transformation & Screening Protocols disclosed in MATCHAMAKER Two-Hybrid System 3 User Manual p. 22 from CLONTECH Co. Transformed cells TM182-CRE1, TM182-AHK2, TM182-AHK2Δ and TM182-AHK3 were produced by selecting transformed cells that were grown in the DOLU+Gal culture medium based on the fact that leucine auxotrophy disappears in the transformed cells.

### Reference Example 2

### Response of Transformed Cells TM182-CRE1, TM182-AHK2, TM182-AHK2Δ and TM182-AHK3 to Cytokinin (Part 1)

A culture solution 10 μl (about 800 clones of yeast) of the transformed cells TM182-CRE1, TM182-AHK2, TM182-AHK2Δ and TM182-AHK3 produced by the example 12 was spotted on DOLU+Glu agar media containing 10 μM of trans-zeatin and cultured at 30° C. for 30 hours. After incubation, the growth state of the transformed cells was observed and photographed by a digital camera.

As a result, each of the transformed cells TM182-CRE1, M182-AHK2, TM182-AHK2Δ and TM182-AHK3 showed high growth in the case of using the DOLU+Glu agar culture media containing trans-zeatin as compared with that in the case of using the DOLU+Glu agar culture media containing no trans-zeatin. The results showed that the transformed cells responded to cytokinin and that they were possible to be grown in the DOLU+Glu agar culture media. Further the transformed cells were found possible to be grown in the DOLU+Gal agar culture media independently of the existence trans-zeatin.

### Reference Example 3

### Response of Transformed Cells TM182-CRE1, TM182-AHK2Δ and TM182-AHK2 to Cytokinin (Part 2)

Culture solutions of the transformed cells TM182-CRE1, TM182-AHK2Δ and TM182-AHK2 produced by the reference example 1 were spotted on DOLU+Glu agar media containing cytokinin in a variety of concentrations (1 nM, 10 nM, 100 nM, 1 μM, 10 μM, 100 μM) and cultured at 30° C. for 30 hours. After incubation, the growth state of the transformed cells was observed and photographed by a digital camera. The lowest observed supply concentrations of trans-zeatin and cis-zeatin in which the respective transformed cells could be grown were shown in Table 1.

TABLE 1

| cytokinin | TM182-CRE1 | TM182-AHK2 | TM182-AHK2Δ |
|---|---|---|---|
| trans-zeatin | 10 μM | 1 μM | 100 nM |
| cis-zeatin | no growth | 10 μM | 1 μM |

### Reference Example 4

### Method for Searching Substance having Agonist-Activity to Cytokinin Receptor (Part 1)

The transformed cells TM182-AHK2, TM182-AHK2Δ and TM182-AHK3 produced in the reference example 1 were inoculated in 200 ml of DOLU+Gal culture media and pre-cultured at 30° C. for 36 hours. The pre-cultured solutions were diluted with DOLU+Glu media as to become 0.100 as the optical density (OD₆₀₀) and further the resultants were diluted with DOLU+Glu media at a dilution rate of 1/200 to obtain diluted pre-cultured solutions.

An assay plate was prepared by filling respective wells of a 96-well plate with 20 μl of solutions which were prepared by diluting DMSO solution (10 mM) of each examinee substance with DOLU+Glu medium at a dilution rate of 1/100, said solutions containing each examinee substance to be 100 μM at the final concentration. Simultaneously, an assay plate only filled with 20 μl of solutions which were prepared by diluting DMSO solution with DOLU+Glu medium, said solutions containing no examinee substance, was prepared as the blank sections.

The diluted pre-cultured solutions were added in 200 μl each into the respective wells of both assay plates and cultured at 30° C. for 24 hours and then the optical density (OD₆₂₀) of each well was measured by a plate reader. The agonist-activity of the examinee substances to the cytokinin receptor was detected by comparing the optical density measured in the testing sections to which the examinee substances were added with the optical density measured in the blank sections. The optical density of the culture solutions of the transformed cells in the testing sections to which the examinee substances were added were shown in Table 2. Compound B showing the higher optical density measured in the testing sections to which the examinee substances were added than that in the blank sections were selected as agonist-active substance to the cytokinin receptor.

TABLE 2

| Examinee | | | |
|---|---|---|---|
| substance | TM182-AHK2 | TM182-AHK2Δ | TM182-AHK3 |
| DMSO | 0.01 | 0 | 0.51 |
| Compound A*¹ | 0.01 | 0 | 0.54 |
| Compound B*² | 0.45 | 0.89 | 0.88 |

*¹Abscisic acid (=negative control)
*²6-Benzyl aminopurine (=positive control)

### Reference Example 5

### Method for Searching Substance having Agonist-Activity to Cytokinin Receptor (Part 2)

The transformed cells TM182-AHK2, TM182-AHK2Δ and M182-AHK3 produced in the reference example 1 are inoculated in 200 ml of DOLU+Gal culture media and cultured at 30° C. for 30 hours to obtain pre-cultured solutions.

The pre-cultured transformed cells (TM182-AHK2, TM182-AHK2Δ and TM182-AHK3) are spot-added in 10 μl to the respective DOLU+Glu agar culture media to which agonist-active substances to the cytokinin receptor selected by the reference example 4 are added while their concentration being changed from 10 nM to 100 μM and then are cultured at 30° C. for 30 hours. After incubation, the intensity of the agonist-activity of the examinee substances to the cytokinin receptor is detected and confirmed based on the lowest concentration at which transformed cells (TM182-AHK2,TM182-AHK2Δ and TM182-AHK3) are observed to grow.

### Example 12

### Preparation of a Polynucleotide Comprising a Nucleotide Sequence Encoding an Amino Acid Sequence of a Cytokinin Biosynthesis Enzyme and Preparation of an Expression Vector of the Polynucleotide: Cloning of AtIPT4 cDNA)

Seeds of Arabidopsis thaliana ecotype Wassilewskija were sterilized with 70% ethyl alcohol for 1 minute, and were further sterilized with 1.5% sodiumhypochlorite for 10 minutes. These seeds were well washed with sterilized water, and then were cultured for 20 days in GM culture medium (4.3 g Murashige and Skoog's basal salt mixture, 1% sucrose, 10 mL of 5% MES-KOH (pH 5.7), 0.3% PHYTAGEL™ (gellangum) (SIGMA)) to obtain 5 g of plants. The plants were frozen in liquefied nitrogen, and were then physically milled in a 1.5 mL tube. To the milled product, 100 μL of 2×CTAB (2% (w/v) Cetyl trimethyl ammonium bromide (hereinafter, referred to as “CTAB”), 0.1M Tris-HCl (pH 8.0), 1.4M NaCl, 1% (w/v) PVP) was added. The resulting mixture was left standing at 60° C. for 30 minutes, and then 100 μL of a mixed solution of chloroform and isoamyl alcohol (chloroform: isoamyl alcohol=24: 1) was added. The resulting mixture was stirred with a Voltex mixer, and was then subjected to centrifugal separation at 15,000 rpm at 20° C. for 20 minutes. After centrifugal separation, 75 μL of the resulting supernatant was placed in a 0.6 mL tube, in which 75 μL of isoamyl alcohol had been already placed, and the resulting mixture was left standing at 25° C. for 20 minutes. Thereafter, the mixture was subjected to centrifugal separation at 15,000 rpm at 20° C. for 20 minutes to recover a precipitate. To the recovered precipitate, 300 μL of 70% ethanol was added, and the resulting mixture was stirred with a Voltex mixer. The mixture was subjected to centrifugal separation at 15,000 rpm at 4° C. for 15 minutes to recover a precipitate. The recovered precipitate was left standing at 37° C. for 5 minutes, and then 30 μL of T10E0.2 (10 mM Tris-HCl (pH 8.0), 0.2 mM EDTA (pH 8.0)) was added to dissolve the precipitate. In this way, a genomic DNA solution was obtained. Next, PCR was carried out using the obtained genomic DNA as a template and a polynucleotide having the nucleotide sequence represented by SEQ ID No: 35 and a polynucleotide having the nucleotide sequence represented by SEQ ID No: 36 as primers to amplify DNA having the nucleotide sequence represented by SEQ ID No: 11. The PCR was carried out employing Herculase Enhanced DNA Polymerase (Stratagene Co., Ltd.) under the amplifying conditions of keeping at 94° C. for 1 minute, repeating 40 cycles each including keeping at 94° C. for 30 seconds, at 50° C. for 30 seconds, and at 72° C. for 60 seconds, and final keeping at 72° C. for 1 minute. A PCR solution (50 μL) was prepared by adding a reaction composition such as dNTP and the like to 100 ng of the template DNA and 100 ng of each of the primers according to the instruction of the kit. In this way, a desired DNA fragment was amplified.

### Example 13

### Preparation of a Polynucleotide Comprising a Nucleotide Sequence Encoding an Amino Acid Sequence of a Cytokinin Biosynthesis Enzyme and Preparation of an Expression Vector of the Polynucleotide: Construction of AtIPT4 Expression Vector

After p423ADH, a yeast expression vector, (Munberg et al. Gene: 156119-122 (1995), available from ATCC library (No.87371) was digested with a restriction enzyme Sma I and then using T4 DNA ligase, DNA having the nucleotide sequence represented by SEQ ID No: 11 and obtained by the example 12 was ligated downstream of the ADH promoter sequence of the expression vector p423ADH as to be integrated to express the desired protein in yeast. The constructed DNA was confirmed to be in the right direction and its nucleotide sequence was confirmed to be the nucleotide sequence represented by SEQ ID No: 11 by an automatic DNA sequencer and then the expression plasmid p423ADH-IPT4 was obtained.

In a similar manner, after p423CYC1, a yeast expression vector, (Munberg et al. Gene: 156119-122 (1995), available from ATCC library (No.87379) was digested with a restriction enzyme Sma I and then using T4 DNA ligase, DNA having the nucleotide sequence represented by SEQ ID No: 11 and obtained by the example 12 was ligated downstream of the CYC 1 promoter sequence of the expression vector p423CYC1 as to be integrated to express the desired protein in yeast. The constructed DNA was confirmed to be in the right direction and its nucleotide sequence was confirmed to be the nucleotide sequence represented by SEQ ID No: 11 by an automatic DNA sequencer and then the expression plasmid p423CYC1-IPT4 was obtained.

### Example 14

### Preparation of a Polynucleotide Comprising a Nucleotide Sequence Encoding an Amino Acid Sequence of a Cytokinin Biosynthesis Enzyme and Preparation of an Expression Vector of the Polynucleotide: Cloning of AtIPT5 cDNA

In a similar manner to Example 12, PCR was carried out by using the genomic DNA obtained from the plants of Arabidopsis thaliana ecotype Wassilewskija as a template and a polynucleotide having a nucleotide sequence represented by SEQ ID No: 37 and polynucleotide having a nucleotide sequence represented by SEQ ID No: 38 as primers to amplify DNA having a nucleotide sequence represented by SEQ ID No: 13. The PCR was carried out employing Herculase Enhanced DNA Polymerase (Stratagene Co., Ltd.) under the amplifying conditions of keeping at 94° C. for 1 minute, repeating 35 cycles each including keeping at 94° C. for 30 seconds, at 55° C. for 30 seconds, and at 72° C. for 90 seconds, and final keeping at 72° C. for 1 minute. A PCR solution (50 μL) was prepared by adding a reaction composition such as dNTP and the like to 100 ng of the template DNA and 100 ng of each of the primers according to the introduction of the kit. In this way, a desired DNA fragment was amplified.

### Example 16

### Production of Transformed Cells of the Present Invention: Production of Transformed Cells TM182-CYC1-CYC1IPT4, TM182-CYC1-ADHIPT4, TM182-CYC1-CYC1IPT5, TM182-CYC1-ADHIPT5, TM182-CRE1-CYC1IPT4, TM182-CRE1-ADHIPT4, TM182-CRE1-CYC1IPT5, TM182-CRE1-ADHIPT5, TM182-AHK2-CYC1IPT4, TM182-AHK2-ADHIPT4, TM182-AHK2-CYC1IPT5, and TM182-AHK2-ADHIPT5

Transformation of TM182 (Sln1Δ), SLN1 genetically defected strain, [Maeda T et al. Nature: 369 242-245 (1994)] was carried out by using any one of the three expression plasmids, commercially available p415CYC1, p415CYC-CRE1 obtained in Example 5, and p415CYC-AHK2 obtained in Example 11, and any one of the four expression plasmids, p423ADH-IPT4 and p423CYC1-IPT4 obtained in Example 13, and p423ADH-IPT5 and p423CYC1-IPT5 obtained in Example 15 together (that is, by using two expression plasmids at the same time). The transformation was carried out by employing Polyethylene glycol/lithium acetate (PEG/LiAc)-mediated transformation method according to the description of VII. Library Transformation & Screening Protocols disclosed in MATCHAMAKER Two-Hybrid System 3 User Manual p. 22 from CLONTECH Co. Twelve kinds of transformed cells TM182-CYC1-CYC1IPT4 to which p415CYC1 and p423CYC1-IPT4 are introduced, TM182-CYC1-ADHIPT4 to which p415CYC1 and p423ADH-IPT4 are introduced, TM182-CYC1-CYC1IPT5 to which p415CYC1 and p423CYC1-IPT5 are introduced, TM182-CYC1-ADHIPT5 to which p415CYC1 and p423ADH-IPT5 are introduced, TM182-CRE1-CYC1IPT4 to which p415CYC-CRE1 and p423CYC1-IPT4 are introduced. TM182-CRE1-ADHIPT4 to which p415CYC-CRE1 and p423ADH-IPT4 are introduced, TM182-CRE1-CYC1IPT5 to which p415CYC-CRE1 and p423CYC1-IPT5 are introduced, TM182-CRE1-ADHIPT5 to which p415CYC-CRE1 and p423ADH-IPT5 are introduced, TM182-AHK2-CYC1IPT4 to which p415CYC-AHK2 and p423CYC1-IPT4 are introduced, TM182-AHK2-ADHIPT4 to which p415CYC-AHK2 and p423ADH-IPT4 are introduced, TM182-AHK2-CYC1IPT5 to which p415CYC-ARK2 and p423CYC1-IPT5 are introduced, and TM182-AHK2-ADHIPT5 to which p415CYC-AHK2 and 423ADH-IPT5 are introduced were produced by selecting transformed cells that were grown in DOHLU+Gal culture medium based on the fact that leucine auxotrophy and histidine auxotrophy disappear in the transformed cells.

### Example 17

### Production of Transformed Cells of the Present Invention: Production of Transformed Cells TM182-AHK2Δ-CYC1IPT4, TM182-AHK2Δ-ADHIPT4, TM182-AHK2Δ-CYC1IPT5, TM182-AHK2Δ-ADHIPT5. TM182-AHK3-CYC1IPT4, TM182-AHK3-ADHIPT4, TM182-AHK3-CYC1IPT5, and TM182-AHK3-ADHIPT5

Transformation of TM182 (Sln1Δ), SLN1 genetically defected strain, [Maeda T et al. Nature: 369 242-245 (1994)] is carried out by using any one of the two expression plasmids, p415CYC-AHK2Δ obtained in Example 11 and p415CYC-AHK3 obtained in Example 8, and any one of the four expression plasmids, p423ADH-IPT4 and p423CYC1-IPT4 obtained in Example 13, and p423ADH-IPT5 and p423CYC1-IPT5 obtained in Example 15 together (that is, by using two expression plasmids at the same time). The transformation is carried out by employing Polyethylene glycol/lithium acetate (PEG/LiAc)-mediated transformation method according to the description of VII. Library Transformation & Screening Protocols disclosed in MATCHAMAKER Two-Hybrid System 3 User Manual p. 22 from CLONTECH Co. Eight kinds of transformed cells TM182-AHK2Δ-CYC1IPT4 to which h415CYC-AHK2Δ and p423CYC1-IPT4 are introduced, TM182-AHK2Δ-ADHIPT4 to which p415CYC-AHK2Δ and p423ADH-IPT4 are introduced, TM182-AHK2Δ-CYC1IPT5 to which p415CYC-AHK2Δ and p423CYC1-IPT5 are introduced, TM182-AHK2Δ-ADHIPT5 to which p415CYC-AHK2Δ and p423ADH-IPT5 are introduced, TM182-AHK3-CYC1IPT4 to which p415CYC-AHK3 and p423CYC1-IPT4 are introduced, TM182-AHK3-ADHIPT4 to which p415CYC-AHK3 and p423ADH-IPT4 are introduced, TM182-AHK3-CYC1IPT5 to which p415CYC-AHK3 and p423CYC1-IPT5 are introduced, and TM182-AHK3-ADHIPT5 to which p415CYC-AHK3 and p423ADH-IPT5 are introduced are produced by selecting transformed cells that are grown in DOHLU+Gal culture medium based on the fact that leucine auxotrophy and histidine auxotrophy disappear in the transformed cells.

### Example 18

### Use of Transformed Cells of the Present Invention: Culture of Transformed Cells TM182-CYC1-CYC1IPT4, TM182-CYC1-ADHIPT4, TM182-CYC1-CYC1IPT5, TM182-CYC1-ADHIPT5, TM182-CRE1-CYC1IPT4, TM182-CRE1-ADHIPT4, TM182-AHK2-CYC1IPT4, TM182-AHK2-ADHIPT4, TM182-AHK2-CYC1IPT5, and TM182-AHK2-ADHIPT5

10 μL of the culture solution (containing about 800 clones of yeast) of each of the transformed cells TM182-CYC1-CYC1IPT4, TM182-CYC1-ADHIPT4, TM182-CYC1-CYC1IPT5, TM182-CYC1-ADHIPT5, TM182-CRE1-CYC1IPT4, TM182-CRE1-ADHIPT4, TM182-AHK2-CYC1IPT4, TM182-AHK2-ADHIPT4, TM182-AHK2-CYC1IPT5, and TM182-AHK2-ADHIPT5 produced in Example 16 was spotted on DOHLU+Glu agar culture medium, and was cultured at 30° C. for 30 hours. After incubation, the growth state of the transformed cells was observed and photographed by a digital camera.

As a result, the transformed cells TM182-CYC1-CYC1IPT4, TM182-CYC1-ADHIPT4, TM182-CYC1-CYC1IPT5, and TM182-CYC1-ADHIPT5 were not observed to grow, but the transformed cells TM182-CRE1-CYC1IPT4, TM182-CRE1-ADHIPT4, TM182-AHK2-CYC1IPT4, TM182-AHK2-ADHIPT4, TM182-AHK2-CYC1IPT5, and TM182-AHK2-ADHIPT5 were observed to grow. Further, in a case where DOHLU+Glu agar culture medium containing 10 μM trans-zeatin was used, the transformed cells TM182-CYC1-CYC1IPT4, TM182-CYC1-ADHIPT4, TM182-CYC1-CYC1IPT5, and TM182-CYC1-ADHIPT5 were not observed to grow, but the transformed cells TM182-CRE1-CYC1IPT4, TM182-CRE1-ADHIPT4, TM182-AHK2-CYC1IPT4, TM182-AHK2-ADHIPT4, TM182-AHK2-CYC1IPT5, and TM182-AHK2-ADHIPT5 were observed to grow. This indicates that the transformed cells TM182-CRE1-CYC1IPT4, TM182-CRE1-ADHIPT4, TM182-AHK2-CYC1IPT4, TM182-AHK2-ADHIPT4, TM182-AHK2-CYC1IPT5, and TM182-AHK2-ADHIPT5 respond to cytokinins synthesized by themselves and can grow on DOHLU+Glu agar culture medium. It was also confirmed that all of the ten kinds of transformed cells could grow in DOHLU+Gal culture medium regardless of the presence or absence of trans-zeatin.

### Example 19

### Use of Transformed Cells of the Present Invention: Culture of Transformed Cells TM182-CYC1-CYC1IPT4, TM182-CYC1-ADHIPT4, TM182-CYC1-CYC1IPT5, TM182-CYC1-ADHIPT5, TM182-CRE1-CYC1IPT4, and TM182-CRE1-ADHIPT5

Each of the transformed cells TM182-CYC1-CYC1IPT4, TM182-CYC1-ADHIPT4, TM182-CYC1-CYC1IPT5, TM182-CYC1-ADHIPT5, TM182-CRE1-CYC1IPT4, and TM182-CRE1-ADHIPT5 obtained in Example 16 was inoculated in 200 mL of DOHLU+Gal culture medium, and was pre-cultured at 30° C. for 36 hours. The thus obtained culture solutions were diluted with DOHLU+Glu culture medium so that OD₆₀₀ became 0.200, and then the diluted ones were further diluted with DOHLU+Glu culture medium at a dilution rate of 1/200 to obtain diluted pre-cultured solutions.

A 96-well assay plate was prepared by filling each well with 1 μL of a DMSO solution of an examinee substance (Abscisic acid or 6-Benzyl aminopurine). In this regard, it is to be noted that the DMSO solution of a examinee substance was prepared so that the concentration thereof became 200 ppm. Simultaneously, an assay plate was prepared by filling each well with 1 μL of DMSO as a blank section.

Each of the diluted pre-cultured solutions was added in a volume of 100 μL per each well of both of the assay plates, and was cultured at 30° C. for 24 hours. After incubation, the turbidity of each well was measured using a plate reader. The absorbance (OD₆₀₀) of each of the culture solutions of the transformed cells containing the examinee substance was shown in Table 3. The transformed cells TM182-CYC1-CYC1IPT4, TM182-CYC1-ADHIPT4, TM182-CYC1-CYC1IPT5, and TM182-CYC1-ADHIPT5 were not observed to grow whichever examinee substance was added. On the other hand, the transformed cells TM182-CRE1-CYC1IPT4 and TM182-CRE1-ADHIPT5 were observed to grow whichever examinee substance was added. Further, the growth speeds of the transformed cells TM182-CRE1-CYC1IPT4 and TM182-CRE1-ADHIPT5 in the culture medium to which 6-Benzyl aminopurine was added were faster than those in the culture medium to which only DMSO was added. This indicates that the transformed cells TM182-CRE1-CYC1IPT4 and TM182-CRE1-ADHIPT5 respond to cytokinins synthesized by the cells and can grow in DOHLU+Glu culture medium and that the growth speeds of the transformed cells TM182-CRE1-CYC1IPT4 and TM182-CRE1-ADHIPT5 are increased or decreased depending on the amount of cytokinin.

TABLE 3

| | TM182- | TM182- | TM182- | TM182- | TM182- | TM182- |
|---|---|---|---|---|---|---|
| | CYC1- | CYC1- | CYC1- | CYC1- | CRE1- | CRE1- |
| Examinee | CYC1- | ADH- | CYC1- | ADH- | CYC1- | ADH- |
| substance | IPT4 | IPT4 | IPT5 | IPT5 | IPT4 | IPT5 |
| DMSO | 0.042 | 0.040 | 0.041 | 0.043 | 0.106 | 0.129 |
| Abscisic | 0.042 | 0.040 | 0.041 | 0.043 | 0.102 | 0.131 |
| acid | | | | | | |
| 6-Benzyl | 0.042 | 0.041 | 0.043 | 0.043 | 0.260 | 0.293 |
| aminopurine | | | | | | |

### Example 20

### Use of Transformed Cells of the Present Invention: Culture of Transformed Cells TM182-CYC1-CYC1IPT4, TM182-CYC1-ADHIPT4, TM182-CYC1-CYC1IPT5, TM182-CYC1-ADHIPT5, TM182-CRE1-ADHIPT4, TM182-AHK2-CYC1IPT4, TM182-AHK2-CYC1IPT5 and TM182-AHK2-ADHIPT5

Each of the transformed cells TM182-CYC1-CYC1IPT4, TM182-CYC1-ADHIPT4, TM182-CYC1-CYC1IPT5, TM182-CYC1-ADHIPT5, TM182-CRE1-ADHIPT4, TM182-AHK2-CYC1IPT4, TM182-AHK2-CYC1IPT5 and TM182-AHK2-ADHIPT5 obtained in Example 16 was inoculated in 200 mL of DOHLU+Gal culture medium, and was pre-cultured at 30° C. for 36 hours. The thus obtained culture solutions were diluted with DOHLU+Glu culture medium so that OD₆₀₀ became 0.200, and then the diluted ones were further diluted with DOHLU+Glu culture medium at a dilution rate of 1/200 to obtain diluted pre-cultured solutions.

A 96-well assay plate was prepared by filling each well with 1 μL of a DMSO solution of an examinee substance (Abscisic acid or 6-Benzyl aminopurine). In this regard, it is to be noted that the DMSO solution of a examinee substance was prepared so that the concentration thereof became 200 ppm. Simultaneously, an assay plate was prepared by filling each well with 1 μL of DMSO as a blank section.

Each of the diluted pre-cultured solutions was added in a volume of 100 μL per each well of both of the assay plates, and was cultured at 30° C. for 24 hours. After incubation, the turbidity of each well was measured using a plate reader. The absorbance (OD₆₀₀) of each of the culture solutions of the transformed cells containing the examinee substance was shown in Table 4. The transformed cells TM182-CYC1-CYC1IPT4, TM182-CYC1-ADHIPT4, TM182-CYC1-CYC1IPT5 and TM182-CYC1-ADHIPT5 were not observed to grow whichever examinee substance was added. On the other hand, the transformed cells TM182-CRE1-ADHIPT4, TM182-AHK2-CYC1IPT4, TM182-AHK2-CYC1IPT5 and TM182-AHK2-ADHIPT5 were observed to grow whichever examinee substance was added. Further, the growth speeds of the transformed cells TM182-CRE1-ADHIPT4, TM182-AHK2-CYC1IPT4, TM182-AHK2-CYC1IPT5 and TM182-AHK2-ADHIPT5 in the culture medium to which 6-Benzyl aminopurine was added were faster than those in the culture medium to which only DMSO was added. This indicates that the transformed cells TM182-CRE1-ADHIPT4, TM182-AHK2-CYC1IPT4, TM182-AHK2-CYC1IPT5 and TM182-AHK2-ADHIPT5 respond to cytokinins synthesized by the cells and can grow in DOHLU+Glu culture medium and that the growth speeds of the transformed cells TM182-CRE1-ADHIPT4, TM182-AHK2-CYC1IPT4, TM182-AHK2-CYC1IPT5 and TM182-AHK2-ADHIPT5 are increased or decreased depending on the amount of cytokinin.

TABLE 4

| | TM182- | TM182- | TM182- | TM182- | TM182- | TM182- | TM182- | TM182- |
|---|---|---|---|---|---|---|---|---|
| | CYC1- | CYC1- | CYC1- | CYC1- | CRE1- | AHK2- | AHK2- | AHK2- |
| Examinee | CYC1- | ADH- | CYC1- | ADH- | ADH- | CYC1- | CYC1- | ADH- |
| substance | IPT4 | IPT4 | IPT5 | IPT5 | IPT4 | IPT4 | IPT5 | IPT5 |
| DMSO | 0.035 | 0.035 | 0.035 | 0.035 | 0.044 | 0.199 | 0.071 | 0.301 |
| Abscisic | 0.035 | 0.035 | 0.036 | 0.035 | 0.045 | 0.194 | 0.069 | 0.297 |
| acid | | | | | | | | |
| 6-Benzyl | 0.034 | 0.034 | 0.035 | 0.035 | 0.058 | 0.339 | 0.430 | 0.427 |
| aminopurine | | | | | | | | |

### Example 21

### Use of Transformed Cells of the Present Invention: Method for Searching Substances having Activity of Controlling Cytokinin Biosynthesis

The transformed cell TM182-CRE1-ADHIPT5 obtained in Example 16 is inoculated in 200 mL of DOHLU+Gal culture medium, and is then pre-cultured at 30° C. for 36 hours. The culture solution is diluted with DOHLU+Glu culture medium so that OD₆₀₀ become 0.200. The diluted one is further diluted with DOHLU+Glu culture medium at a dilution ratio of 1/200 to obtain a diluted pre-cultured solution. Similarly, TM182 (sln1 Δ), a SLN1 genetically defected strain in which PTP2 Tyrosine phosphatase gene is introduced, is inoculated in 200 mL of DOLU+Gal culture medium, and is then pre-cultured at 30° C. for 36 hours. The culture solution is diluted with DOLU+Gal culture medium so that OD₆₀₀ become 0.100. The diluted one is further diluted with DOLU+Gal culture medium at a dilution ratio of 1/200 to obtain a diluted pre-cultured solution. Further, the transformed cell TM182-CRE1 obtained in Reference Example 1 is inoculated in 200 mL of DOLU+Gal culture medium, and is then pre-cultured at 30° C. for 36 hours. The culture solution is diluted with DOLU+Glu culture medium so that OD₆₀₀ become 0.100. The diluted one is further diluted with DOLU+Glu culture medium at a dilution ratio of 1/200 to obtain a diluted pre-cultured solution. A diluted pre-cultured solution of transformed cell TM182-CRE1 to which 6-Benzyl aminopurine (cytokinin) is added is also prepared so that the concentration of the 6-Benzyl aminopurine become 2 ppm.

A 96-well assay plate is prepared by filling each well with 1 μL of a DMSO solution of a examinee substance. In this regard, it is to be noted that the DMSO solution of a examinee substance is prepared so that the concentration thereof become 200 ppm. Simultaneously, an assay plate is prepared by filling each well with 1 μL of DMSO as a blank section.

Each of the diluted pre-cultured solutions is added in a volume of 100 μL per each well of both of the assay plates, and is cultured at 30° C. for 24 hours. After incubation, the turbidity of each well is measured using a plate reader. In a case where it is confirmed that the growth speed of the transformed cell TM182-CRE1-ADHIPT5 in a well containing a examinee substance is increased or decreased as compared with that in a well of the blank section to which only DMSO is added, the examinee substance contained in the well can be selected as a substance having the activity of controlling cytokinin biosynthesis. Incidentally, in a case where, in a well containing the examinee substance which is confirmed to increase or decrease the growth speed of the transformed cell TM182-CRE1-ADHIPT5 as compared with that in the blank section to which only DMSO is added, the growth speed of the transformed cell TM182-CRE1 is not increased or decreased as compared with that in the blank section to which only DMSO is added, the examinee substance can be selected as a substance that has the activity of controlling cytokinin biosynthesis and that may not directly affect signal transduction system from cytokinin receptor. Further, in a case where, in a well containing the examinee substance which is confirmed to increase or decrease the growth speed of the transformed cell TM182-CRE1-ADHIPT5 as compared with that in the blank section to which only DMSO is added, the growth speed of the transformed cell TM182 (sln1 Δ) is not increased or decreased as compared with that in the blank section to which only DMSO is added, the examinee substance can be selected as a substance that has the activity of controlling cytokinin biosynthesis and that may not affect somewhere not involved in biosynthesis of cytokinin nor signal transduction from cytokinin receptor.

Hereinafter, the medium compositions to be used in the present invention will be described:

(a) DOLU+Glu culture medium

| | |
|---|---|
| Bacto-yeast nitrogen base without amino acids | 6.7 g |
| Glucose | 20 g |
| Drop-out mix (x) | 2.0 g |
| Distilled water | 1000 ml |

(b) DOLU+Gal culture medium

| | |
|---|---|
| Bacto-yeast nitrogen base without amino acids | 6.7 g |
| Galactose | 20 g |
| Drop-out mix (x) | 2.0 g |
| Distilled water | 1000 ml |

(x) Drop-out mix: Drop-out mix is a combination of the following ingredients.

| | | | |
|---|---|---|---|
| Adenine | 0.5 g | Lysine | 2.0 g |
| Alanine | 2.0 g | Methionine | 2.0 g |
| Arginine | 2.0 g | para-Aminobenzoic | 0.2 g |
| | | acid | |
| Asparagine | 2.0 g | Phenylalanine | 2.0 g |
| Aspartic acid | 2.0 g | Proline | 2.0 g |
| Cysteine | 2.0 g | Serine | 2.0 g |
| Glutamine | 2.0 g | Threonine | 2.0 g |
| Glutamic acid | 2.0 g | Trytophan | 2.0 g |
| Glycine | 2.0 g | Tyrosine | 2.0 g |
| Histidine | 2.0 g | Valine | 2.0 g |
| Inositol | 2.0 g | Isoleucine | 2.0 g |

(c) DOLU+Glu agar culture medium

A solid culture medium prepared by adding 2% (W/V) of agar into the culture medium (a)

(d) DOLU+Gal agar culture medium

A solid culture medium prepared by adding 2% (W/V) of agar into the culture medium (c)

(e) DOHLU+Glu culture medium

| | |
|---|---|
| Bacto-yeast nitrogen base without amino acids | 6.7 g |
| Glucose | 20 g |
| Drop-out mix (y) | 2.0 g |
| Distilled water | 1000 ml |

(f) DOHLU+Gal culture medium

| | |
|---|---|
| Bacto-yeast nitrogen base without amino acids | 6.7 g |
| Galactose | 20 g |
| Drop-out mix (y) | 2.0 g |
| Distilled water | 1000 ml |

(y) Drop-out mix: Drop-out mix is a combination of the following ingredients.

| | |
|---|---|
| Adenine | 0.5 g |
| Alanine | 2.0 g |
| Arginine | 2.0 g |
| Asparagine | 2.0 g |
| Aspartic acid | 2.0 g |
| Cysteine | 2.0 g |
| Glutamine | 2.0 g |
| Glutamic acid | 2.0 g |
| Glycine | 2.0 g |
| Inositol | 2.0 g |
| Isoleucine | 2.0 g |
| Lysine | 2.0 g |
| Methionine | 2.0 g |
| para-Aminobenzoic | 0.2 g |
| acid | |
| Phenylalanine | 2.0 g |
| Proline | 2.0 g |
| Serine | 2.0 g |
| Threonine | 2.0 g |
| Trytophan | 2.0 g |
| Tyrosine | 2.0 g |
| Valine | 2.0 g |

(g) DOHLU+Glu agar culture medium

A solid culture medium prepared by adding 2% (W/V) of agar into the culture medium (e)

(h) DOHLU+Gal agar culture medium

A solid culture medium prepared by adding 2% (W/V) of agar into the culture medium (f)
Free text in Sequence Listing SEQ ID No: 23
Designed oligonucleotide primer for PCR SEQ ID No: 24
Designed oligonucleotide primer for PCR SEQ ID No: 25
Designed oligonucleotide primer for PCR SEQ ID No: 26
Designed oligonucleotide primer for PCR SEQ ID No: 27
Designed oligonucleotide primer for PCR SEQ ID No: 28
Designed oligonucleotide primer for PCR SEQ ID No: 29
Designed oligonucleotide primer for PCR SEQ ID No: 30
Designed oligonucleotide primer for PCR SEQ ID No: 31
Designed oligonucleotide primer for PCR SEQ ID No: 32
Designed oligonucleotide primer for PCR SEQ ID No: 33
Designed oligonucleotide primer for PCR SEQ ID No: 34
Designed oligonucleotide primer for PCR SEQ ID No: 35
Designed oligonucleotide primer for PCR SEQ ID No: 36
Designed oligonucleotide primer for PCR SEQ ID No: 37
Designed oligonucleotide primer for PCR SEQ ID No: 38
Designed oligonucleotide primer for PCR

1. A cell transformed with a polynucleotide comprising a nucleotide sequence encoding an amino acid sequence of a cytokinin receptor and a polynucleotide comprising a nucleotide sequence encoding an amino acid sequence of a cytokinin biosynthesis enzyme.

2. The transformed cell according to claim 1, wherein said cytokinin receptor is selected from the group consisting of:
(a) a cytokinin receptor having the amino acid sequence represented by SEQ ID No: 2;
(b) a cytokinin receptor having the amino acid sequence represented by SEQ ID No: 4;
(c) a cytokinin receptor having the amino acid sequence represented by SEQ ID No: 6;
(d) a partially transmembrane region-deleted type cytokinin receptor;
(e) a cytokinin receptor having the amino acid sequence represented by amino acids 196 to 1176 of SEQ ID No: 4;
(f) a cytokinin receptor having the amino acid sequence represented by amino acids 50 to 1176 of SEQ ID No: 4;
(g) a cytokinin receptor having the amino acid sequence represented by amino acids 32 to 1036 of SEQ ID No: 6;
(h) a chimera-type cytokinin receptor comprising an extracellular region of the cytokinin receptor, transmembrane regions of the cytokinin receptor, a histidine kinase region of the cytokinin receptor, and a receiver region of the histidine kinase, wherein said extracellular region, said transmembrane regions and said histidine kinase region are homogeneous to one another and the receiver region is heterogeneous thereto;
(i) a cytokinin receptor having the amino acid sequence of (a), (b), (c), (e), (f), or (g) with deletion, substitution, or addition of one or a plurality of amino acids; and
(j) a cytokinin receptor having an amino acid sequence encoded by a nucleotide sequence of a polynucleotide, wherein said polynucleotide hybridizes under a stringent condition to a polynucleotide that has a nucleotide sequence complementary to a nucleotide sequence encoding the amino acid sequence represented by SEQ ID Nos: 2, 4, or 6.

3. The transformed cell according to claim 1, wherein said cytokinin biosynthesis enzyme is a isopentenyltransferase.

4. The transformed cell according to claim 1, wherein said cytokinin biosynthesis enzyme is selected from the group consisting of:
(a) a cytokinin biosynthesis enzyme having the amino acid sequence represented by SEQ ID No: 8;
(b) a cytokinin biosynthesis enzyme having the amino acid sequence represented by SEQ ID No: 10;
(c) a cytokinin biosynthesis enzyme having the amino acid sequence represented by SEQ ID No: 12;
(d) a cytokinin biosynthesis enzyme having the amino acid sequence represented by SEQ ID No: 14;
(e) a cytokinin biosynthesis enzyme having the amino acid sequence represented by SEQ ID No: 16;
(f) a cytokinin biosynthesis enzyme having the amino acid sequence represented by SEQ ID No: 18;
(g) a cytokinin biosynthesis enzyme having the amino acid sequence represented by SEQ ID No: 20;
(h) a cytokinin biosynthesis enzyme having the amino acid sequence represented by SEQ ID Nos: 8, 10, 12, 14, 16, 18, or 20 with deletion, substitution, or addition of one or a plurality of amino acids; and
(i) a cytokinin biosynthesis enzyme having an amino acid sequence encoded by a nucleotide sequence of a polynucleotide, wherein said polynucleotide hybridizes under a stringent condition to a polynucleotide that has a nucleotide sequence complementary to a nucleotide sequence encoding the amino acid sequence represented by SEQ ID Nos: 8, 10, 12, 14, 16, 18, or 20.

5. The transformed cell according to claim 1, wherein said cell is yeast.

6. The transformed cell according to claim 1, wherein said cell is budding yeast.
